(19) United States
(12) Patent Application Publication
(10) Pub. No.: US 2026/0176335 A1
(43) Pub. Date: Jun. 25, 2026

(54) ANTIBODIES TARGETING SERUM AMYLOID A (SAA) AND USES THEREOF

(71) Applicant: The Trustees of Columbia University in the City of New York, New York, NY (US)

(72) Inventors: Stavroula KOUSTENI, New York, NY (US); Marta GALÁN-DÍEZ, New York, NY (US); Pallavi M. BUDGUDE, New York, NY (US); Brian CHERNAK, New York, NY (US)

(73) Assignee: The Trustees of Columbia University in the City of New York, New York, NY (US)

(21) Appl. No.: 19/532,667

(22) Filed: Feb. 6, 2026

Related U.S. Application Data
(63) Continuation of application No. PCT/US24/42391, filed on Aug. 15, 2024.
(60) Provisional application No. 63/519,621, filed on Aug. 15, 2023.

(51) Int. Cl. C07K 16/00; C07K 16/28

(52) U.S. Cl. CPC C07K 16/00; C07K 16/2896; C07K 2317/24; C07K 2317/55; C07K 2317/56; C07K 2317/565; C07K 2317/76; C07K 2317/92

(57) ABSTRACT

The present disclosure relates to anti-SAA antibodies or fragments thereof, such as antibodies against human SAA1 or fragments thereof, that may be used in various therapeutic, prophylactic and diagnostic methods. The present antibodies or fragments thereof may be used to treat hematologic disorders such as acute myeloid leukemia, acute lymphoid leukemia, and myelodysplastic syndrome.

### CROSS REFERENCE TO RELATED APPLICATIONS

This application is a continuation application of International Patent Application No. PCT/US2024/042391, filed on Aug. 15, 2024, which claims priority to U.S. Provisional Application No. 63/519,621, filed on Aug. 15, 2023, the disclosure of each of which is incorporated herein by reference in its entirety.

### GOVERNMENT SUPPORT

This invention was made with government support under AR054447 and HL130937 awarded by the National Institutes of Health. The government has certain rights in the invention.

### SEQUENCE LISTING

The application contains a sequence listing which has been submitted electronically in XML format and is hereby incorporated by reference in its entirety. Said XML copy, created on Aug. 12, 2024, is named 01001_012072-WO0_SL.xml and is 51,526 bytes in size.

### FIELD OF THE DISCLOSURE

The present disclosure relates to the treatment of leukemia and myelodysplastic syndrome via the inhibition of serum amyloid A1 (SAA1), as well as anti-SAA1 antibodies.

### BACKGROUND OF THE DISCLOSURE

Acute myeloid leukemia (AML) is a type of blood cancer characterized by two features: inability to form mature red blood cells and immune cells, as well as excessive number of dysfunctional white blood cells which influence the immune response. It often emerges from pre-AML states of clonal hematopoiesis (CH) and myelodysplastic syndrome (MDS). MDS and AML patients usually have various diseases of the immune system, high levels of inflammatory proteins in their bone marrow and a suppressed immune system.

Recent studies indicate that the tumor microenvironment plays an important role in disease development. For example, osteoblasts are cells important for the formation of new bones. Osteoblast numbers are decreased in MDS and AML patients and their ablation increases leukemia burden, whereas maintaining the osteoblastic pool reduces tumor burden and prolongs survival. Alterations in the osteoblastic compartment can lead to MDS and AML in mice, and are associated with myeloproliferative neoplasms, MDS and AML in patients. In addition, osteoblasts can exert a tumor-suppressor role in myeloid disorders or can be remodeled by dysplastic cells to reinforce leukemia.

AML progression requires the presence of serotonin receptor-1b (HTR1B) in osteoblasts and is driven by AML-secreted kynurenine, which acts as an oncometabolite and HTRIB ligand. AML cells utilize kynurenine to induce a pro-inflammatory state in osteoblasts which, through the acute-phase protein serum amyloid A (SAA), acts in a positive feedback-loop on leukemia cells by increasing expression of indoleamine 2,3-dioxygenase (IDO1), a rate-limiting enzyme for kynurenine synthesis, thereby enabling AML progression.

Standard of care (SOC) therapy relies on hypomethylating agents for MDS and chemotherapy for AML and some new targeted therapies including targeting IDH1 or IDH2 inhibitors. However, their efficacy is short-lived and not curative. Moreover, 30% of MDS patients transform to AML at which point the disease becomes largely unresponsive to treatments and highly lethal. In addition to targeting tumor cells directly, there is a need for therapies directed against other causes of AML, such as the tumor microenvironment (Krevvata M, et al., Inhibition of leukemia cell engraftment and disease progression in mice by osteoblasts. Blood. 2014 October; 124 (18): pp. 2834-46). Specifically, osteoblasts can be targeted to inhibit leukemia engraftment and disease progression (Krevvata M, et al., Inhibition of leukemia cell engraftment and disease progression in mice by osteoblasts. Blood. 2014 October; 124 (18): pp. 2834-46).

Therefore, new therapies that limit the number of cancer cells in the blood or lessen the suppression of the immune system are needed.

### SUMMARY

The present disclosure provides for an anti-SAA antibody, or an antigen-binding portion thereof, comprising a heavy chain variable region (V_{H}) and a light chain variable region (V_{L}), wherein the heavy chain variable region comprises three complementarity determining regions (CDRs), CDR1, CDR2 and CDR3, having amino acid sequences about 80% to about 100% identical to the amino acid sequences set forth in SEQ ID NOs: 8, 9 and 10, respectively, SEQ ID NOs: 42, 43 and 44, respectively, SEQ ID NOs: 11, 12 and 13, respectively, or SEQ ID NOs: 14, 15 and 16, respectively; and wherein the light chain variable region comprises three CDRs, CDR1, CDR2 and CDR3, having amino acid sequences about 80% to about 100% identical to the amino acid sequences set forth in SEQ ID NOs: 24, 25 and 26, respectively, or SEQ ID NOs: 52, 53 and 54, respectively (or SEQ ID NO: 27, the sequence of WTS and SEQ ID NO: 29, respectively).

The present disclosure provides for an anti-SAA antibody, or an antigen-binding portion thereof, comprising a heavy chain variable region (V_{H}), wherein the heavy chain variable region comprises three CDRs, CDR1, CDR2 and CDR3, having amino acid sequences about 80% to about 100% identical to the amino acid sequences set forth in SEQ ID NOs: 8, 9 and 10, respectively, SEQ ID NOs: 42, 43 and 44, respectively, SEQ ID NOs: 11, 12 and 13, respectively, or SEQ ID NOs: 14, 15 and 16, respectively.

The present disclosure provides for an anti-SAA antibody, or an antigen-binding portion thereof, comprising a light chain variable region (V_{L}), wherein the light chain variable region comprises three CDRs, CDR1, CDR2 and CDR3, having amino acid sequences about 80% to about 100% identical to the amino acid sequences set forth in SEQ ID NOs: 24, 25 and 26, respectively, or SEQ ID NOs: 52, 53 and 54, respectively (or SEQ ID NO: 27, the sequence of WTS and SEQ ID NO: 29, respectively).

The dissociation constant (K_{D}) of the antibody, or antigen-binding portion thereof, may be less than about 2×10⁻⁹ M, less than about 1.5×10⁻⁹ M, or less than about 1.2×10⁻⁹ M.

The heavy chain variable region (V_{H}) of the antibody or antigen-binding portion thereof may comprise an amino acid sequence about 80% to about 100% identical to the amino acid sequences set forth in SEQ ID NOs: 6, 7, 40, or 41. The light chain variable region (V_{L}) of the antibody or antigen-binding portion thereof may comprise an amino acid sequence about 80% to about 100% identical to the amino acid sequences set forth in SEQ ID NOs: 22, 23, 50, or 51.

The antibody or antigen-binding portion thereof may be selected from the group consisting of: (a) a whole immunoglobulin molecule; (b) an scFv; (c) a Fab fragment; (d) an F(ab′)2; and (e) a disulfide linked Fv.

The antibody or antigen-binding portion thereof may comprise at least one constant domain selected from the group consisting of: a) an IgG constant domain; and (b) an IgA constant domain.

The antibody or antigen-binding portion thereof may comprise at least one human constant domain.

The antibody or antigen-binding portion thereof may bind to a peptide having an amino acid sequence about 80% to about 100% identical to the amino acid sequences set forth in SEQ ID NOs: 32, 33 or 34.

The present disclosure provides for an anti-SAA antibody, or an antigen-binding portion thereof, comprising a heavy chain variable region (V_{H}) and a light chain variable region (V_{L}), wherein the heavy chain variable region comprises three complementarity determining regions (CDRs), CDR1, CDR2 and CDR3, encoded by nucleotide sequences about 80% to about 100% identical to the nucleotide sequences set forth in SEQ ID NOs: 3, 4 and 5, respectively, or SEQ ID NOs: 37, 38 and 39, respectively; and wherein the light chain variable region comprises three CDRs, CDR1, CDR2 and CDR3, encoded by nucleotide sequences about 80% to about 100% identical to the nucleotide sequences set forth in SEQ ID NOs: 19, 20 and 21, respectively or SEQ ID NOs: 47, 48 and 49, respectively.

The present disclosure provides for an anti-SAA antibody, or an antigen-binding portion thereof, comprising a heavy chain variable region (V_{H}) and a light chain variable region (V_{L}), wherein the heavy chain variable region is encoded by a nucleotide sequence about 80% to about 100% identical to the nucleotide sequences set forth in SEQ ID NOs: 1, 2, 35, or 36, and wherein the light chain variable region is encoded by a nucleotide sequence about 80% to about 100% identical to the nucleotide sequences set forth in SEQ ID NOs: 17, 18, 45, or 46.

The SAA may be human SAA1 or mouse SAA3.

The antibody or antigen-binding portion thereof may be humanized or chimeric.

The present disclosure also provides for an isolated polypeptide comprising the present antibody or antigen-binding portion thereof.

Also encompassed by the present disclosure is a composition comprising the antibody or antigen-binding portion thereof, and at least one pharmaceutically acceptable carrier.

The present disclosure provides for a polynucleotide encoding the antibody or antigen-binding portion thereof. The present disclosure provides for a vector comprising the polynucleotide. The present disclosure also provides for a cell comprising the vector.

Also encompassed by the present disclosure is a method of treating a cancer or a hematologic disorder in a subject, the method comprising administering to the subject an effective amount of the antibody or antigen-binding portion thereof, the polypeptide, or the composition.

The hematologic disorder may be acute myeloid leukemia (AML), myelodysplastic syndrome (MDS), chronic myelomonocytic leukemia (CMML), or acute lymphoid leukemia (ALL).

The cancer may be pancreatic cancer, lung cancer, liver cancer, breast cancer or colon cancer.

The subject may be a human.

The administration may be parenteral, intravenous, subcutaneous, intramuscular, transdermal, oral, topical, intrathecal, or local.

The method may further comprise administering to the subject a chemotherapeutic agent, an inhibitor of indoleamine 2,3 dioxygenase (IDO1), an inhibitor of kynurenine synthesis, an agent facilitating kynurenine breakdown, an inhibitor of PD1, an inhibitor of PD-L1, or combinations thereof.

The present disclosure provides for an anti-SAA1 antibody or an antigen-binding portion thereof, wherein the anti-SAA1 antibody or antigen-binding portion binds to a linear amino acid sequence epitope comprising the amino acid sequence set forth in SEQ ID NO:33 or SEQ ID NO:32.

The present disclosure provides for a method of treating myelodysplastic syndrome or acute myelogenous leukemia in a subject, the method comprising administering to the subject a therapeutically effective amount of an anti-SAA1 antibody or an antigen-binding portion thereof, wherein the anti-SAA1 antibody or antigen-binding portion thereof binds to a linear amino acid sequence epitope comprising the amino acid sequence set forth in SEQ ID NO:33 or SEQ ID NO:32.

### BRIEF DESCRIPTION OF THE DRAWINGS

FIGS. 1A-1C: Effects of the anti-SAA3 polyclonal antibody. FIG. 1A: Inhibition of SAA3-induced NFκB activation in RAW 264.7 cells by the anti-SAA3 polyclonal antibody. FIG. 1B: Effect of the anti-SAA3 polyclonal antibody on leukemia progression in mice injected with MLL-AF9 cells. FIG. 1C: Effect of anti-SAA3 polyclonal antibody on survival in mice injected with MLL-AF9 cells.

FIG. 2: Synthetic peptide immunization strategy. The peptide used for immunization and ELISA screening is a modified SAA1/SAA3 amino acid 33-42 peptide Ac-RDMWRAYSDMC-amide.

FIGS. 3A-3D: Evaluation of antibody subclones in inhibiting SAA1-induced NFκB activation and cell viability.

FIGS. 4A-4F: Competitive binding assay using a reporter cell line.

FIGS. 5A-5B: Inhibition of SAA1-induced proliferation by the anti-SAA1 monoclonal antibody in cancer cell lines using EdU incorporation assay. FIG. 5A: The purified 18A3 antibodies effectively inhibited SAA1-induced proliferation in T-cell acute lymphoblastic leukemia (T-ALL) cell lines (CUTLL1, CEM and Jurkat), and B-cell acute lymphoblastic leukemia (B-ALL) cell lines (Nalm6, REH and 697). FIG. 5B: The purified 18A3 antibodies effectively inhibited SAA1-induced proliferation in pancreatic cancer cell lines (BXPC3, HPAC and PANC1).

### DETAILED DESCRIPTION

The present disclosure relates to anti-SAA antibodies and antibody fragments (e.g., antigen-binding portions of the antibody) that may be used in various therapeutic, prophylactic, diagnostic and other methods. The present antibodies or fragments thereof, or the present composition, may be used to treat a cancer or a hematologic disorder in a subject.

Applications of the present antibodies (or fragments thereof) and compositions include (i) treatments for a cancer or a hematologic disorder (e.g., AML or myelodysplasia), (ii) combination therapy with chemotherapies/immunotherapies for a cancer or a hematologic disorder (e.g., AML or myelodysplasia), (iii) modulating bone marrow niche interactions in the context of stem cell transplantation and immunodeficiency disorders, and (iv) improving the in vitro culturing of hematopoietic stem cells.

The antibodies, or antigen-binding portions thereof, include, but are not limited to, monoclonal antibodies, humanized antibodies, chimeric antibodies, polyclonal antibodies, recombinantly expressed antibodies, as well as antigen-binding portions of the foregoing. An antigen-binding portion of an antibody may include a portion of an antibody that specifically binds to SAA (e.g., SAA1).

The present disclosure also provides for a method for treating a cancer or a hematologic disorder. The method may comprise administering a therapeutically effective amount of an anti-SAA (e.g., anti-SAA1) antibody or its fragment to a subject in need thereof. The method may comprise administering a composition to a subject in need thereof. The composition may comprise the anti-SAA (e.g., anti-SAA1) antibody or fragments thereof. The hematologic disorder may be myelodysplastic syndrome (MDS) or leukemia (e.g., acute myeloid leukemia (AML), acute lymphocytic leukemia (or acute lymphoblastic leukemia, ALL)).

In one embodiment, the present disclosure provides for a method of treating or ameliorating myelodysplastic syndrome (MDS) or leukemia in a subject. The method may comprise administering to the subject a composition comprising the present antibody (or its fragment) in an amount sufficient to decrease one or more of the symptoms of myelodysplastic syndrome (MDS) or leukemia in the subject.

In another embodiment, the antibody or antigen-binding fragment is administered to a subject having myelodysplastic syndrome (MDS), or leukemia such as acute myeloid leukemia (AML), and acute lymphocytic leukemia (or acute lymphoblastic leukemia, ALL), in a subject.

Also encompassed by the present disclosure is a method of blocking the function of SAA (e.g., SAA1) in a subject. The method may comprise administering to the subject a composition comprising the present antibodies, or antigen-binding portions thereof, in an amount sufficient to decrease or block at least one of the SAA-mediated functions in the subject.

The present antibody or antigen-binding portion thereof, or the present composition, may be used to prevent or decrease (the rate or likelihood of) age-related clonal hematopoiesis (ARCH) transformation to MDS, or MDS transformation to AML.

In addition to the present antibody or its fragment, the present methods may further comprise administering a second therapeutic agent such as a chemotherapeutic agent, an inhibitor of indoleamine 2,3 dioxygenase (IDO1), an inhibitor of kynurenine synthesis, an agent facilitating kynurenine breakdown, an inhibitor of PD1, an inhibitor of PD-L1, or combinations thereof.

The present antibodies, or antigen-binding portions thereof, may specifically bind to human SAA1 and/or mouse SAA3, including recombinant and native human SAA1.

An antibody light or heavy chain variable region consists of three hypervariable regions, referred to as complementarity determining regions (CDRs). CDRs are supported within the variable regions by framework regions (FRs). In one embodiment, the heavy chain variable region (or light chain variable region) contains three CDRs and four framework regions (FRs), arranged from amino-terminus to carboxyl-terminus in the following order: FR1, CDR1, FR2, CDR2, FR3, CDR3, FR4. Kabat, E. A., et al. Sequences of Proteins of Immunological Interest, Fifth Edition, U.S. Department of Health and Human Services, NIH Publication No. 91-3242, 1991. Chothia, C. et al., J. Mol. Biol. 196:901-917, 1987.

### Variable Regions and CDRs

The heavy chain variable regions, light chain variable regions and CDRs of the murine anti-SAA1 4B10 antibody, are shown below.

Heavy chain variable region V_{H} (including signal sequence): DNA sequence (414 bp)
Signal sequence-FR1-CDR1-FR2-CDR2-FR3-CDR3-FR4 (SEQ ID NO: 1):
ATGGATTGGCTGTGGAACTTGCTATTCCTGATGGCAGCTGCCCAAAGTGCCCAAGCAC
AGATCCAGTTGGTGCAGTCTGGACCTGAGCTGAGGAAGCCTGGAGAGACAGTCA
AGATCTCCTGCAAGGCTTCTGGATATACCTTCACAAACTATGGATTGAACTGGA
TTAAGCAGGCTCCAGGAAAGGGTTTACAGTGGATGGGCTGGATAAACACCTAC
ACTGGAAAGCCAACGTATGCTGATGAATTCAAGGAGCGATTTGTCTTCTCTTT
GGACACCTTTGCCCGCACTGCCTATTTACAGATTAATAACCTCAAAAATGATGAC
ATGGCAACATATTTCTGTGCAAAATCCCTCCGACGGGACAGGCACTTTGACTA
CTGGGGCCAAGGCATCTCTCTCACAGTCTCCTCA
Heavy chain variable region V_{H}: DNA sequence
FR1-CDR1-FR2-CDR2-FR3-CDR3-FR4 (SEQ ID NO: 2):
CAGATCCAGTTGGTGCAGTCTGGACCTGAGCTGAGGAAGCCTGGAGAGACAGTC
AAGATCTCCTGCAAGGCTTCTGGATATACCTTCACAAACTATGGATTGAACTGG
ATTAAGCAGGCTCCAGGAAAGGGTTTACAGTGGATGGGCTGGATAAACACCTA
CACTGGAAAGCCAACGTATGCTGATGAATTCAAGGAGCGATTTGTCTTCTCTT
TGGACACCTTTGCCCGCACTGCCTATTTACAGATTAATAACCTCAAAAATGATGA
CATGGCAACATATTTCTGTGCAAAA TCCCTCCGACGGGACAGGCACTTTGACT
ACTGGGGCCAAGGCATCTCTCTCACAGTCTCCTCA
DNA encoding HCDR1 (heavy chain CDR1): AACTATGGATTGAAC (SEQ ID NO: 3)
DNA encoding HCDR2 (heavy chain CDR2):
TGGATAAACACCTACACTGGAAAGCCAACGTATGCTGATGAATTCAAGGAG
(SEQ ID NO: 4)
DNA encoding HCDR3 (heavy chain CDR3):
TCCCTCCGACGGGACAGGCACTTTGACTAC (SEQ ID NO: 5)
Heavy chain variable region V_{H} (including signal sequence): Amino acid sequence (138 aa)
Signal peptide-FR1-CDR1-FR2-CDR2-FR3-CDR3-FR4 (SEQ ID NO: 6):
MDWLWNLLFLMAAAQSAQAQIQLVQSGPELRKPGETVKISCKASGYTFTNYGLNWIK
QAPGKGLQWMGWINTYTGKPTYADEFKERFVFSLDTFARTAYLQINNLKNDDMA
TYFCAKSLRRDRHEDYWGQGISLTVSS
Heavy chain variable region V_{H}: Amino acid sequence
FR1-CDR1-FR2-CDR2-FR3-CDR3-FR4 (SEQ ID NO: 7):
QIQLVQSGPELRKPGETVKISCKASGYTFTNYGLNWIKQAPGKGLQWMGWINTYT
GKPTYADEFKERFVFSLDTFARTAYLQINNLKNDDMATYFCAKSLRRDRHFDYWG
QGISLTVSS
Kabat system:
HCDR1: NYGLN (SEQ ID NO: 8)
HCDR2: WINTYTGKPTYADEFKE (SEQ ID NO: 9)
HCDR3: SLRRDRHFDY (SEQ ID NO: 10)
Clothia system:
HCDR1: GYTFTNY (SEQ ID NO: 11)
HCDR2: NTYTGK (SEQ ID NO: 12)
HCDR3: SLRRDRHFDY (SEQ ID NO: 13)
IMGT system:
HCDR1: GYTFTNYG (SEQ ID NO: 14)
HCDR2: INTYTGKP (SEQ ID NO: 15)
HCDR3: AKSLRRDRHFDY (SEQ ID NO: 16)
Light chain variable region V_{L} (including signal sequence): DNA sequence (381 bp)
Signal sequence-FR1-CDR1-FR2-CDR2-FR3-CDR3-FR4 (SEQ ID NO: 17):
ATGGAGACACATTCTCAGGTCTTTGTATACATGTTGCTGTGGTTGTCTGGTGTTGAAGG
AGACATTGTGATGACCCAGTCTCACAAATTCATGTCCGCATCAGTCGGAGACAGG
GTCAGCATCACCTGCAAGGCCAGTCAGGATGTAGATACTGCTGTAGCCTGGTA
TCAACAGAAACCACGACACTCTCCTAAACTTCTGATTTATTGGACATCCACCCG
ACACACT
GGAGTCCCTGATCGCTTCACAGGCAGTGGATCTGGGACAGATTTCACTCTCACCA
TTAGCAATGTGCAGTCTGAAGACTTGGCAGATTATTTCTGTCAGCAATTTAACA
CCTATCCTCTCACGTTCGGTACTGGGACCAAGGTGGAGCTGAAA
Light chain variable region V_{L}: DNA sequence
FR1-CDR1-FR2-CDR2-FR3-CDR3-FR4 (SEQ ID NO: 18):
GACATTGTGATGACCCAGTCTCACAAATTCATGTCCGCATCAGTCGGAGACAGG
GTCAGCATCACCTGCAAGGCCAGTCAGGATGTAGATACTGCTGTAGCCTGGTA
TCAACAGAAACCACGACACTCTCCTAAACTTCTGATTTATTGGACATCCACCCG
ACACACTGGAGTCCCTGATCGCTTCACAGGCAGTGGATCTGGGACAGATTTCAC
TCTCACCATTAGCAATGTGCAGTCTGAAGACTTGGCAGATTATTTCTGTCAGCAA
TTTAACACCTATCCTCTCACGTTCGGTACTGGGACCAAGGTGGAGCTGAAA
DNA encoding LCDR1 (light chain CDR1):
AAGGCCAGTCAGGATGTAGATACTGCTGTAGCC (SEQ ID NO: 19)
DNA encoding LCDR2 (light chain CDR2):
TGGACATCCACCCGACACACT (SEQ ID NO: 20)
DNA encoding LCDR3 (light chain CDR3):
CAGCAATTTAACACCTATCCTCTCACG (SEQ ID NO: 21)
Light chain variable region V_{L} (including signal peptide): Amino acid sequence (127 aa)
Signal peptide-FR1-CDR1-FR2-CDR2-FR3-CDR3-FR4 (SEQ ID NO: 22):
METHSQVEVYMLLWLSGVEGDIVMTQSHKFMSASVGDRVSITCKASQDVDTAVAWY
QQKPRHSPKLLIYWTSTRHTGVPDRFTGSGSGTDFTLTISNVQSEDLADYFCQQFNT
YPLTFGTGTKVELK
Light chain variable region V_{L}: Amino acid sequence
FR1-CDR1-FR2-CDR2-FR3-CDR3-FR4 (SEQ ID NO: 23):
DIVMTQSHKFMSASVGDRVSITCKASQDVDTAVAWYQQKPRHSPKLLIYWTSTRH
TGVPDRFTGSGSGTDFTLTISNVQSEDLADYFCQQFNTYPLTFGTGTKVELK
Kabat system (Clothia system):
LCDR1: KASQDVDTAVA (SEQ ID NO: 24)
LCDR2: WTSTRHT (SEQ ID NO: 25)
LCDR3: QQFNTYPLT (SEQ ID NO: 26)
IMGT system:
LCDR1: QDVDTA (SEQ ID NO: 27)
LCDR2: WTS
LCDR3: QQFNTYPLT (SEQ ID NO: 29)

The heavy chain variable regions, light chain variable regions and CDRs of the murine anti-SAA1 18A3 antibody, are shown below.

Heavy chain variable region V_{H} (including signal sequence): DNA sequence (420 bp)
Signal sequence-FR1-CDR1-FR2-CDR2-FR3-CDR3-FR4 (SEQ ID NO: 35):
ATGGACTCCAGGCTCAATTTAGTTTTCCTTGTCCTTATTTTAAAAGGTGTCCAGTGTGAT
GTGCAGCTGGTGGAGTCTGGGGGAGGCTTAGTGCAGTCTGGAGGGTCCCGGAAA
CTCTCCTGTGCAGCCTCTGGATTCACTTTCAGTAGGTTTGGAATGCACTGGGTTC
GTCAGGCTCCAGAGAAGGGGCTGGAGTGGGTCGCATACATTAGTCGTGGCAGT
ACTAAAATCTACTATGCAGACACAGTGAAGGGCCGATTCACCATCTCCAGAGA
CAATCCCAGGAACACCCTGTTCCTGCAAATGACCAGTCTAAGGTCTGAGGACAC
GGCCATGTATTACTGTGCAAGATCTTTGATCTACTATGATTACGACGGTTTTG
GTTACTGGGGCCAAGGGACTCTGGTCACTGTCTCTGCA
Heavy chain variable region VH: DNA sequence
FR1-CDR1-FR2-CDR2-FR3-CDR3-FR4 (SEQ ID NO: 36):
GATGTGCAGCTGGTGGAGTCTGGGGGAGGCTTAGTGCAGTCTGGAGGGTCCCGG
AAACTCTCCTGTGCAGCCTCTGGATTCACTTTCAGTAGGTTTGGAATGCACTGG
GTTCGTCAGGCTCCAGAGAAGGGGCTGGAGTGGGTCGCATACATTAGTCGTGG
CAGTACTAAAATCTACTATGCAGACACAGTGAAGGGCCGATTCACCATCTCCA
GAGACAATCCCAGGAACACCCTGTTCCTGCAAATGACCAGTCTAAGGTCTGAGG
ACACGGCCATGTATTACTGTGCAAGATCTTTGATCTACTATGATTACGACGGT
TTTGGTTACTGGGGCCAAGGGACTCTGGTCACTGTCTCTGCA
DNA encoding HCDR1: AGGTTTGGAATGCAC (SEQ ID NO: 37)
DNA encoding HCDR2:
TACATTAGTCGTGGCAGTACTAAAATCTACTATGCAGACACAGTGAAGGGC (SEQ
ID NO: 38)
DNA encoding HCDR3: TCTTTGATCTACTATGATTACGACGGTTTTGGTTAC (SEQ
ID NO: 39)
Heavy chain variable region V_{H} (including signal sequence): Amino acid sequence (140 aa)
Signal peptide-FR1-CDR1-FR2-CDR2-FR3-CDR3-FR4 (SEQ ID NO: 40):
MDSRLNLVELVLILKGVQCDVQLVESGGGLVQSGGSRKLSCAASGFTFSRFGMHWVR
QAPEKGLEWVAYISRGSTKIYYADTVKGRFTISRDNPRNTLFLQMTSLRSEDTAMY
YCARSLIYYDYDGFGYWGQGTLVTVSA
Heavy chain variable region V_{H}: Amino acid sequence
FR1-CDR1-FR2-CDR2-FR3-CDR3-FR4 (SEQ ID NO: 41):
DVQLVESGGGLVQSGGSRKLSCAASGFTFSRFGMHWVRQAPEKGLEWVAYISRGS
TKIYYADTVKGRFTISRDNPRNTLFLQMTSLRSEDTAMYYCARSLIYYDYDGFGYW
GQGTLVTVSA
HCDR1: RFGMH (SEQ ID NO: 42)
HCDR2: YISRGSTKIYYADTVKG (SEQ ID NO: 43)
HCDR3: SLIYYDYDGFGY (SEQ ID NO: 44)
Light chain variable region V_{L} (including signal sequence): DNA sequence (393 bp)
Signal sequence-FR1-CDR1-FR2-CDR2-FR3-CDR3-FR4 (SEQ ID NO: 45):
ATGAAGTTGCCTGTTAGGCTGTTGGTGCTGATGTTCTGGATTCCTGCTTCCAGCAGT
GATGTTTTGATGACCCAAACTCCACTCTCCCTGCCTCTCAGTCTTGGAGATCAAGC
CTCCATCTCTTGCAGATCTAGTCAGAGCATTGTACATAGTAATGGAAACACCT
ATTTAGAATGGTACCTGCAGAAACCAGGCCAGTCTCCAAAGCTCCTGATCTACA
AAGTTTCCAACCGATTTTCTGGGGTCCCAGACAGGTTCAGTGGCAGTGGATCA
GGGACAGATTTCACACTCAAGATCAGCAGAGTGGAGGCTGAGGATCTGGGAGTT
TATTACTGCTTTCAAGGTTCACATGTTCCTCCGACGTTCGGTGGAGGCACCAA
GCTGGAAATCAAA
Light chain variable region V_{L}: DNA sequence
FR1-CDR1-FR2-CDR2-FR3-CDR3-FR4 (SEQ ID NO: 46):
GATGTTTTGATGACCCAAACTCCACTCTCCCTGCCTCTCAGTCTTGGAGATCAAG
CCTCCATCTCTTGCAGATCTAGTCAGAGCATTGTACATAGTAATGGAAACACC
TATTTAGAATGGTACCTGCAGAAACCAGGCCAGTCTCCAAAGCTCCTGATCTAC
AAAGTTTCCAACCGATTTTCTGGGGTCCCAGACAGGTTCAGTGGCAGTGGATC
AGGGACAGATTTCACACTCAAGATCAGCAGAGTGGAGGCTGAGGATCTGGGAGT
TTATTACTGCTTTCAAGGTTCACATGTTCCTCCGACGTTCGGTGGAGGCACCAA
GCTGGAAATCAAA
DNA encoding LCDR1:
AGATCTAGTCAGAGCATTGTACATAGTAATGGAAACACCTATTTAGAA (SEQ ID
NO: 47)
DNA encoding LCDR2: AAAGTTTCCAACCGATTTTCT (SEQ ID NO: 48)
DNA encoding LCDR3: TTTCAAGGTTCACATGTTCCTCCGACG (SEQ ID NO: 49)
Light chain variable region V_{L} (including signal peptide): Amino acid sequence (131 aa)
Signal peptide-FR1-CDR1-FR2-CDR2-FR3-CDR3-FR4 (SEQ ID NO: 50):
MKLPVRLLVLMFWIPASSSDVLMTQTPLSLPLSLGDQASISCRSSQSIVHSNGNTYLEW
YLQKPGQSPKLLIYKVSNRFSGVPDRFSGSGSGTDFTLKISR VEAEDLGVYYCFQGS
HVPPTFGGGTKLEIK
Light chain variable region V_{L}: Amino acid sequence
FR1-CDR1-FR2-CDR2-FR3-CDR3-FR4 (SEQ ID NO: 51):
DVLMTQTPLSLPLSLGDQASISCRSSQSIVHSNGNTYLEWYLQKPGQSPKLLIYKVS
NRFSGVPDRFSGSGSGTDFTLKISRVEAEDLGVYYCFQGSHVPPTFGGGTKLEIK
LCDR1: RSSQSIVHSNGNTYLE (SEQ ID NO: 52)
LCDR2: KVSNRFS (SEQ ID NO: 53)
LCDR3: FQGSHVPPT (SEQ ID NO: 54)

In certain embodiments, the antibody or antigen-binding portion thereof includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 6, SEQ ID NO: 7, SEQ ID NO: 40, or SEQ ID NO: 41.

In certain embodiments, the antibody or antigen-binding portion thereof includes a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 22, SEQ ID NO: 23, SEQ ID NO: 50, or SEQ ID NO: 51.

In certain embodiments, the antibody or antigen-binding portion thereof includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 6, SEQ ID NO: 7, SEQ ID NO: 40, or SEQ ID NO: 41; and a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 22, SEQ ID NO: 23, SEQ ID NO: 50, or SEQ ID NO: 51.

A heavy chain variable region (V_{H}) of the antibody or antigen-binding portion thereof can comprise one, two, three or more complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 4B10 antibody (CDR1, CDR2 and CDR3 set forth in SEQ ID NOs: 8, 9, 10, respectively; or CDR1, CDR2 and CDR3 set forth in SEQ ID NOs: 11, 12, 13, respectively; or CDR1, CDR2 and CDR3 set forth in SEQ ID NOs: 14, 15, 16, respectively).

A heavy chain variable region (V_{H}) of the antibody or antigen-binding portion thereof can comprise one, two, three or more complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 18A3 antibody (CDR1, CDR2 and CDR3 set forth in SEQ ID NOs: 42, 43, 44, respectively).

A light chain variable region (V_{L}) of the antibody or antigen-binding portion thereof can comprise one, two, three or more CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 4B10 antibody (CDR1, CDR2 and CDR3 set forth in SEQ ID NOs: 24, 25, 26, respectively; or CDR1, CDR2 and CDR3 set forth in SEQ ID NO: 27, the sequence of WTS, SEQ ID NO: 29, respectively).

A light chain variable region (V_{L}) of the antibody or antigen-binding portion thereof can comprise one, two, three or more CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 18A3 antibody (CDR1, CDR2 and CDR3 set forth in SEQ ID NOs: 52, 53, 54, respectively).

A heavy chain variable region (V_{H}) of the present antibody, or antigen-binding portion thereof, can comprise one, two, three or more complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 4B10 antibody (CDR1, CDR2 and CDR3 set forth in SEQ ID NOs: 8, 9, 10, respectively; or CDR1, CDR2 and CDR3 set forth in SEQ ID NOs: 11, 12, 13, respectively; or CDR1, CDR2 and CDR3 set forth in SEQ ID NOs: 14, 15, 16, respectively), and a light chain variable region (V_{L}) of the antibody or antigen-binding portion thereof can comprise one, two, three or more CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 4B10 antibody (CDR1, CDR2 and CDR3 set forth in SEQ ID NOs: 24, 25, 26, respectively; or CDR1, CDR2 and CDR3 set forth in SEQ ID NO: 27, the sequence of WTS, SEQ ID NO: 29, respectively).

A heavy chain variable region (V_{H}) of the present antibody, or antigen-binding portion thereof, can comprise one, two, three or more complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 18A3 antibody (CDR1, CDR2 and CDR3 set forth in SEQ ID NOs: 42, 43, 44, respectively), and a light chain variable region (V_{L}) of the antibody or antigen-binding portion thereof can comprise one, two, three or more CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 18A3 antibody (CDR1, CDR2 and CDR3 set forth in SEQ ID NOs: 52, 53, 54, respectively).

A heavy chain variable region (V_{H}) of the antibody or antigen-binding portion thereof can include three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 4B10 antibody (CDR1, CDR2 and CDR3 set forth in SEQ ID NOs: 8, 9, 10, respectively; or CDR1, CDR2 and CDR3 set forth in SEQ ID NOs: 11, 12, 13, respectively; or CDR1, CDR2 and CDR3 set forth in SEQ ID NOs: 14, 15, 16, respectively).

A heavy chain variable region (V_{H}) of the antibody or antigen-binding portion thereof can include three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 18A3 antibody (CDR1, CDR2 and CDR3 set forth in SEQ ID NOs: 42, 43, 44, respectively).

In one embodiment, a light chain variable region (V_{L}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 4B10 antibody (CDR1, CDR2 and CDR3 set forth in SEQ ID NOs: 24, 25, 26, respectively; or CDR1, CDR2 and CDR3 set forth in SEQ ID NO: 27, the sequence of WTS, SEQ ID NO: 29, respectively).

In one embodiment, a light chain variable region (V_{L}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 18A3 antibody (CDR1, CDR2 and CDR3 set forth in SEQ ID NOs: 52, 53, 54, respectively).

In one embodiment, a heavy chain variable region (V_{H}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 4B10 antibody (CDR1, CDR2 and CDR3 set forth in SEQ ID NOs: 8, 9, 10, respectively; or CDR1, CDR2 and CDR3 set forth in SEQ ID NOs: 11, 12, 13, respectively; or CDR1, CDR2 and CDR3 set forth in SEQ ID NOs: 14, 15, 16, respectively), and a light chain variable region (V_{L}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 4B10 antibody (CDR1, CDR2 and CDR3 set forth in SEQ ID NOs: 24, 25, 26, respectively; or CDR1, CDR2 and CDR3 set forth in SEQ ID NO: 27, the sequence of WTS, SEQ ID NO: 29, respectively).

In one embodiment, a heavy chain variable region (V_{H}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a heavy chain variable region of the 18A3 antibody (CDR1, CDR2 and CDR3 set forth in SEQ ID NOs: 42, 43, 44, respectively), and a light chain variable region (V_{L}) of the antibody or antigen-binding portion thereof includes three CDRs that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the CDRs of a light chain variable region of the 18A3 antibody (CDR1, CDR2 and CDR3 set forth in SEQ ID NOs: 52, 53, 54, respectively).

In certain embodiments, a heavy chain variable region (V_{H}) of the antibody or antigen-binding portion thereof includes three CDRs that are identical to CDRs of a heavy chain variable region of the 4B10 antibody (CDR1, CDR2 and CDR3 set forth in SEQ ID NOs: 8, 9, 10, respectively; or CDR1, CDR2 and CDR3 set forth in SEQ ID NOs: 11, 12, 13, respectively; or CDR1, CDR2 and CDR3 set forth in SEQ ID NOs: 14, 15, 16, respectively), and a light chain variable region (V_{L}) of the antibody or antigen-binding portion thereof includes three CDRs that are identical to CDRs of a light chain variable region of the 4B10 antibody (CDR1, CDR2 and CDR3 set forth in SEQ ID NOs: 24, 25, 26, respectively; or CDR1, CDR2 and CDR3 set forth in SEQ ID NO: 27, the sequence of WTS, SEQ ID NO: 29, respectively).

In certain embodiments, a heavy chain variable region (V_{H}) of the antibody or antigen-binding portion thereof includes three CDRs that are identical to CDRs of a heavy chain variable region of the 18A3 antibody (CDR1, CDR2 and CDR3 set forth in SEQ ID NOs: 42, 43, 44, respectively), and a light chain variable region (V_{L}) of the antibody or antigen-binding portion thereof includes three CDRs that are identical to CDRs of a light chain variable region of the 18A3 antibody (CDR1, CDR2 and CDR3 set forth in SEQ ID NOs: 52, 53, 54, respectively).

Encompassed by the present disclosure are antibodies with a heavy chain variable region (V_{H}) and a light chain variable region (V_{L}) having amino acid sequences at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the heavy chain variable region (SEQ ID NO: 7 or SEQ ID NO: 6) and light chain variable region (SEQ ID NO: 23 or SEQ ID NO: 22) of the antibody 4B10, respectively.

Encompassed by the present disclosure are antibodies with a heavy chain variable region (V_{H}) and a light chain variable region (V_{L}) having amino acid sequences at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the heavy chain variable region (SEQ ID NO: 41 or SEQ ID NO: 40) and light chain variable region (SEQ ID NO: 51 or SEQ ID NO: 50) of the antibody 18A3, respectively.

In related embodiments, anti-SAA (e.g., anti-SAA1) antibodies or antigen-binding portions thereof include, for example, the CDRs of heavy chain variable regions and/or light chain variable regions of the antibody 4B10 or 18A3.

In one embodiment, the antibody or antigen-binding portion thereof contains a heavy chain variable region and a light chain variable region identical to a heavy chain variable region and light chain variable region of the 4B10 antibody (SEQ ID NO: 7 (or SEQ ID NO: 6), and SEQ ID NO: 23 (or SEQ ID NO: 22), respectively).

In one embodiment, the antibody or antigen-binding portion thereof contains a heavy chain variable region and a light chain variable region identical to a heavy chain variable region and light chain variable region of the 18A3 antibody (SEQ ID NO: 41 (or SEQ ID NO: 40), and SEQ ID NO: 51 (or SEQ ID NO: 50), respectively).

In various embodiments, the antibodies or antigen-binding portions thereof specifically bind to an epitope that overlaps with, or are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to an epitope bound by the 4B10 or 18A3 antibody. The epitope may be present within the sequence of SEQ ID NO: 30, or may be amino acids 33-42 of SEQ ID NO: 30. The epitope may be RDMWRAYSDMC (SEQ ID NO: 32) or RDMWRAYSDM (SEQ ID NO: 33).

In certain embodiments, the antibodies or antigen-binding portions thereof specifically bind to an epitope that overlaps with, or are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to an epitope that is present within the sequence of SEQ ID NO: 30, an epitope that is amino acids 33-42 of SEQ ID NO: 30, an epitope that is RDMWRAYSDMC (SEQ ID NO: 32), or an epitope that is RDMWRAYSDM (SEQ ID NO: 33).

In certain embodiments, the CDRs have sequence variations. For example, CDRs, in which 1, 2 3, 4, 5, 6, 7 or 8 residues, or less than 20%, less than 30%, or less than about 40%, of the total residues in the CDR, are substituted or deleted can be present in an antibody (or antigen-binding portion thereof) that binds SAA (e.g., SAA1).

Also within the scope of the disclosure are antibodies or antigen-binding portions thereof in which specific amino acids have been substituted, deleted or added. These alternations do not have a substantial effect on the peptide's biological properties such as binding activity. For example, antibodies may have amino acid substitutions in the framework region, such as to improve binding to the antigen. In another example, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in Bowie et al., Science, 247:1306-1310 (1990). Cunningham et al., Science, 244:1081-1085 (1989). Ausubel (ed.), Current Protocols in Molecular Biology, John Wiley and Sons, Inc. (1994). T. Maniatis, E. F. Fritsch and J. Sambrook, Molecular Cloning: A Laboratory Manual, Cold Spring Harbor laboratory, Cold Spring Harbor, N.Y. (1989). Pearson, Methods Mol. Biol. 243:307-31 (1994). Gonnet et al., Science 256:1443-45 (1992).

The present peptides may be the functionally active variant of antibodies of antigen-binding portions thereof disclosed herein, e.g., with less than or about 30%, less than or about 25%, less than or about 20%, less than or about 15%, less than or about 10%, less than or about 5%, less than or about 3%, or less than or about 1% amino acid residues substituted or deleted but retain essentially the same immunological properties including, but not limited to, binding to SAA (e.g., SAA1).

The antibodies or antigen-binding portions thereof may also include variants, analogs, orthologs, homologs and derivatives of peptides, that exhibit a biological activity, e.g., binding of an antigen such as SAA (e.g., SAA1). The peptides may contain one or more analogs of an amino acid (including, for example, non-naturally occurring amino acids, amino acids which only occur naturally in an unrelated biological system, modified amino acids from mammalian systems etc.), peptides with substituted linkages, as well as other modifications known in the art.

The antibody, or antigen-binding portion thereof, can be derivatized or linked to another functional molecule. For example, an antibody can be functionally linked (by chemical coupling, genetic fusion, noncovalent interaction, etc.) to one or more other molecular entities, such as another antibody, a detectable agent, an immunosuppressant, a cytotoxic agent, a pharmaceutical agent, a protein or peptide that can mediate association with another molecule (such as a streptavidin core region or a poly-histidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof. Such cytotoxic agents can be coupled to the antibodies of the present disclosure using standard procedures, and used, for example, to treat a patient indicated for therapy with the antibody.

One type of derivatized protein is produced by crosslinking two or more proteins (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

In another embodiment, the anti-SAA (e.g., anti-SAA1) antibody or its fragment is used unlabeled and detected with a labeled antibody that binds the anti-SAA (e.g., anti-SAA1) antibody or its fragment.

### Antibody Fragments

The antibodies can be full-length or can include a fragment (or fragments) of the antibody having an antigen-binding portion, including, but not limited to, Fab, F(ab′)2, Fab′, F (ab)′, Fv, single chain Fv (scFv), bivalent scFv (bi-scFv), trivalent scFv (tri-scFv), Fd, dAb fragment (e.g., Ward et al., Nature, 341:544-546 (1989)), an isolated CDR, diabodies, triabodies, tetrabodies, linear antibodies, single-chain antibody molecules, and multi-specific antibodies formed from antibody fragments. Single chain antibodies produced by joining antibody fragments using recombinant methods, or a synthetic linker, are also encompassed by the present disclosure. Bird et al. Science, 1988, 242:423-426. Huston et al., Proc. Natl. Acad. Sci. USA, 1988, 85:5879-5883.

The antibody or antigen-binding portion thereof may be the following: (a) a whole immunoglobulin molecule; (b) a single-chain variable fragment (scFv); (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

Antibody fragments may be generated by traditional means, such as enzymatic digestion, or by recombinant techniques.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

Fv is the minimum antibody fragment which contains a complete antigen-binding site. In one embodiment, a two-chain Fv species consists of a dimer of one heavy-chain and one light-chain variable domain in tight, non-covalent association. In a single-chain Fv (scFv) species, one heavy-chain and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although maybe at a lower affinity than the entire binding site.

The Fab fragment contains the heavy-chain and light-chain variable domains and also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Single-chain Fv or scFv antibody fragments comprise the V_{H} and V_{L} domains of antibody, where these domains are present in a single polypeptide chain. Generally, the scFv polypeptide further comprises a polypeptide linker between the V_{H} and V_{L} domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

Diabodies are antibody fragments with two antigen-binding sites, which fragments comprise a heavy-chain variable domain (V_{H}) connected to a light-chain variable domain (V_{L}) in the same polypeptide chain (V_{H}-V_{L} Or V_{L}-V_{H}). Diabodies may be bivalent or bispecific. Diabodies are described in, for example, European Patent No. 404,097; PCT Publication WO 1993/01161; Hudson et al., Nat. Med. 9:129-34, 2003; and Hollinger et al., Proc. Natl. Acad. Sci. USA 90:6444-8, 1993. Triabodies and tetrabodies are also described in Hudson et al., Nat. Med. 9:129-34, 2003.

Various techniques have been developed for the production of antibody fragments. Traditionally, these fragments were derived via proteolytic digestion of intact antibodies (see, e.g., Morimoto et al., J. Biochem. Biophys. Methods 24:107-17, 1992; and Brennan et al., Science 229:81-3, 1985). The fragments can also be produced by recombinant techniques. Fab, Fv, and ScFv antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al., Bio/Technology 10:163-7, 1992). In another approach, F(ab′)₂ fragments are isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner.

The present antibody or antigen-binding portion thereof may comprise at least one constant domain, such as, (a) an IgG constant domain; (b) an IgA constant domain, etc.

All antibody isotypes are encompassed by the present disclosure, including IgG (e.g., IgG1, IgG2, IgG3, IgG4), IgM, IgA (IgA1, IgA2), IgD or IgE. The antibodies or antigen-binding portions thereof may be mammalian (e.g., mouse, human) antibodies or antigen-binding portions thereof. The light chains of the antibody may be of kappa or lambda type. An alternative anti-SAA (e.g., anti-SAA1) antibody can comprise sequences from more than one immunoglobulin class or isotype, and selecting particular constant domains to optimize desired effector functions is within the ordinary skill in the art.

The antibodies or antigen-binding portions thereof of the present disclosure may be mono-specific, bi-specific or multi-specific. Multi-specific or bi-specific antibodies or fragments thereof may be specific for different epitopes of one target polypeptide (e.g., SAA such as SAA1) or may contain antigen-binding domains specific for more than one target polypeptide (e.g., antigen-binding domains specific for SAA and other antigen relating to MDS or leukemia). In one embodiment, a multi-specific antibody or antigen-binding portion thereof comprises at least two different variable domains, wherein each variable domain is capable of specifically binding to a separate antigen or to a different epitope on the same antigen. Tutt et al., 1991, J. Immunol. 147:60-69. Kufer et al., 2004, Trends Biotechnol. 22:238-244. The present antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity. For example, the present disclosure includes bi-specific antibodies wherein one arm of an immunoglobulin is specific for SAA (e.g., SAA1), and the other arm of the immunoglobulin is specific for a second therapeutic target or is conjugated to a therapeutic moiety.

### Humanized Antibodies

A humanized antibody of the present disclosure is an antibody from a non-human species where the amino acid sequences in the non-antigen binding regions (and/or the antigen-binding regions) have been altered so that the antibody more closely resembles a human antibody, and still retains its original binding ability.

In certain embodiments, humanized antibodies are antibody molecules from non-human species having one, two, three or all CDRs from the non-human species, and one, two, three, four or all framework regions from a human immunoglobulin molecule.

The CDRs of the present antibodies or antigen-binding portions thereof can be from a non-human or human source. The framework of the present antibodies or antigen-binding portions thereof can be human, humanized, non-human (e.g., a murine framework modified to decrease antigenicity in humans), or a synthetic framework (e.g., a consensus sequence). In one embodiment, the present antibodies, or antigen-binding portions thereof, contain at least one heavy chain variable region and/or at least one light chain variable region.

The humanized antibodies of the present disclosure can be produced by methods known in the art. For example, a humanized antibody can have one or more amino acid residues introduced into it from a source which is non-human. These non-human amino acid residues are often referred to as “import” residues, which are typically taken from an “import” variable domain. Humanization can be performed following the method of Winter and co-workers (Jones et al., Nature 321:522-5, 1986; Riechmann et al., Nature 332:323-7, 1988; Verhoeyen et al., Science 239:1534-6, 1988), by substituting hypervariable region sequences for the corresponding sequences of a human antibody. Accordingly, in such humanized antibodies, substantially less than an intact human variable domain has been substituted by the corresponding sequence from a non-human species. In certain embodiments, humanized antibodies are human antibodies in which at least some hypervariable region residues as well as other variable region residues are substituted by residues from analogous sites in non-human antibodies.

The choice of human variable domains, both light and heavy, to be used in making the humanized antibodies, may reduce antigenicity. According to the “best-fit” method, the sequence of the variable domain of a non-human (e.g., rodent such as mouse) antibody is screened against the entire library of known human variable domain sequences. The human sequence which is closest to that of the non-human is then accepted as the human framework for the humanized antibody. See, e.g., Sims et al., J. Immunol. 151:2296-308, 1993; Chothia et al., J. Mol. Biol. 196:901-17, 1987. Another method uses a particular framework derived from the consensus sequence of all human antibodies of a particular subgroup of light or heavy chains. The same framework may be used for several different humanized antibodies. See, e.g., Carter et al., Proc. Natl. Acad. Sci. USA 89:4285-9, 1992; Presta et al., J. Immunol. 151:2623-32, 1993.

Humanized antibodies can be generated by replacing sequences of the variable region that are not directly involved in antigen binding with equivalent sequences from human variable regions. Those methods include isolating, manipulating, and expressing the nucleic acid sequences that encode all or part of variable regions from at least one of a heavy or light chain. Sources of such nucleic acid are well known to those skilled in the art and, for example, may be obtained from a hybridoma producing an antibody against SAA. The recombinant DNA encoding the humanized antibody, or fragment thereof, can then be cloned into an appropriate expression vector.

In another example, once non-human (e.g., murine) antibodies are obtained, variable regions can be sequenced, and the location of the CDRs and framework residues determined. Kabat, E. A., et al. (1991) Sequences of Proteins of Immunological Interest, Fifth Edition, U.S. Department of Health and Human Services, NIH Publication No. 91-3242. Chothia, C. et al. (1987) J. Mol. Biol., 196:901-917. The light and heavy chain variable regions can, optionally, be ligated to corresponding constant regions. CDR-grafted antibody molecules can be produced by CDR-grafting or CDR substitution. One, two, three or all CDRs of an immunoglobulin chain can be replaced. For example, all of the CDRs of a particular antibody may be from at least a portion of a non-human animal (e.g., mouse such as the CDRs described herein) or only some of the CDRs may be replaced. It is only necessary to keep the CDRs required for binding of the antibody to a predetermined antigen (e.g., SAA1). Morrison, S. L., 1985, Science, 229:1202-1207. Oi et al., 1986, BioTechniques, 4:214. U.S. Pat. Nos. 5,585,089; 5,225,539; 5,693,761 and 5,693,762. EP 519596. Jones et al., 1986, Nature, 321:552-525. Verhoeyan et al., 1988, Science, 239:1534. Beidler et al., 1988, J. Immunol., 141:4053-4060.

It may be desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

In some embodiments, a humanized anti-SAA (e.g., anti-SAA1) antibody also includes at least a portion of an immunoglobulin constant region, e.g., a constant region of a human immunoglobulin. In one embodiment, the antibody will contain both the light chain as well as at least the variable domain of a heavy chain. The antibody also may include one or more of the constant domain CH1, hinge, CH2, CH3, and/or CH4 of the heavy chain, as appropriate.

In some aspects of the present disclosure, one or more domains of the humanized antibodies will be recombinantly expressed. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of humanized anti-SAA (e.g., anti-SAA1) antibody in prokaryotic and eukaryotic host cells.

Also encompassed by the present disclosure are antibodies, or antigen-binding portions thereof, containing one, two, or all CDRs as disclosed herein, with the other regions replaced by sequences from at least one different species including, but not limited to, human, rabbits, sheep, dogs, cats, cows, horses, goats, pigs, monkeys, apes, gorillas, chimpanzees, ducks, geese, chickens, amphibians, reptiles and other animals.

### Human Antibodies

Human antibodies of the disclosure can be constructed by combining Fv clone variable domain sequence(s) selected from human-derived phage display libraries with known human constant domain sequences(s) (Hoogenboom et al., J. Mol. Biol. 227:381-8, 1992; Marks et al., J. Mol. Biol. 222:581-97, 1991). Alternatively, human antibodies can be made by the hybridoma method. Human myeloma and mouse-human heteromyeloma cell lines for the production of human monoclonal antibodies have been described, for example, by Kozbor, J. Immunol. 133:3001-5, 1984; Brodeur et al., Monoclonal Antibody Production Techniques and Applications, pp. 51-63 (Marcel Dekker, Inc., New York, 1987); and Boerner et al., J. Immunol. 147:86-95, 1991.

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551-5, 1993; Jakobovits et al., Nature 362:255-8, 1993; Brüggemann et al., Year Immunol. 7:33-40, 1993.

Gene shuffling can also be used to derive human antibodies from non-human, e.g., rodent, antibodies, where the human antibody has similar affinities and specificities to the starting non-human antibody. According to this method, which is also called “epitope imprinting,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab where the human chain restores the antigen binding site destroyed upon removal of the corresponding non-human chain in the primary phage display clone, i.e., the epitope governs (imprints) the choice of the human chain partner. When the process is repeated in order to replace the remaining non-human chain, a human antibody is obtained (see PCT Publication WO 93/06213). Unlike traditional humanization of non-human antibodies by CDR grafting, this technique provides completely human antibodies, which have no FR or CDR residues of non-human origin.

### Chimeric Antibodies

A chimeric antibody is a molecule in which different portions are derived from different animal species. For example, an antibody may contain a variable region derived from a murine antibody and a human immunoglobulin constant region. Chimeric antibodies can be produced by recombinant DNA techniques. Morrison, et al., Proc Natl Acad Sci, 81:6851-6855 (1984). For example, a gene encoding a murine (or other species) monoclonal antibody molecule is digested with restriction enzymes to remove the region encoding the murine Fc, and the equivalent portion of a gene encoding a human Fc constant region is substituted. Chimeric antibodies can also be created by recombinant DNA techniques where DNA encoding murine V regions can be ligated to DNA encoding the human constant regions. Better et al., Science, 1988, 240:1041-1043. Liu et al. PNAS, 1987 84:3439-3443. Liu et al., J. Immunol., 1987, 139:3521-3526. Sun et al. PNAS, 1987, 84:214-218. Nishimura et al., Canc. Res., 1987, 47:999-1005. Wood et al. Nature, 1985, 314:446-449. Shaw et al., J. Natl. Cancer Inst., 1988, 80:1553-1559. International Patent Publication Nos. WO1987002671 and WO 86/01533. European Patent Application Nos. 184, 187; 171,496; 125,023; and 173,494. U.S. Pat. No. 4,816,567.

### Production of Antibodies

The present disclosure provides for methods for making an antibody or antigen-binding portion thereof that specifically binds to SAA (e.g., SAA1).

For example, a non-human animal is immunized with a composition that includes SAA (e.g., SAA1), or a fragment thereof (e.g., a peptide), and then a specific antibody is isolated from the animal. The method can further include evaluating binding of the antibody to SAA (e.g., SAA1).

In one embodiment, the present disclosure provides for a method for making a hybridoma that expresses an antibody that specifically binds to SAA (e.g., SAA1). The method may contain the following steps: immunizing an animal with a composition that includes SAA (e.g., SAA1) or its fragment; isolating splenocytes from the animal; generating hybridomas from the splenocytes; and selecting a hybridoma that produces an antibody that specifically binds to SAA (e.g., SAA1). Kohler and Milstein, Nature, 256:495, 1975. Harlow, E. and Lane, D. Antibodies: A Laboratory Manual, Cold Spring Harbor Laboratory Press, Cold Spring Harbor, N.Y., 1988.

In one embodiment, SAA (e.g., SAA1) or its fragment is used to immunize mice intraperitoneally or intravenously. One or more boosts may or may not be given. The titers of the antibodies in the plasma can be monitored by, e.g., ELISA (enzyme-linked immunosorbent assay) or flow cytometry. Mice with sufficient titers of anti-SAA (e.g., anti-SAA1) antibodies are used for fusions. Mice may or may not be boosted with antigen 3 days before sacrifice and removal of the spleen. The mouse splenocytes are isolated and fused with PEG to a mouse myeloma cell line. The resulting hybridomas are then screened for the production of antigen-specific antibodies. Cells are plated, and then incubated in selective medium. Supernatants from individual wells are then screened by ELISA for human anti-SAA (e.g., anti-SAA1) monoclonal antibodies. The antibody secreting hybridomas are replated, screened again, and if still positive for anti-SAA (e.g., anti-SAA1) monoclonal antibodies, can be subcloned by limiting dilution.

Adjuvants that may be used to increase the immunogenicity of SAA (e.g., SAA1) or its fragment include any agent or agents that act to increase an immune response to peptides or combination of peptides. Non-limiting examples of adjuvants include alum, aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid, QS21 (saponin adjuvant), MPL (Monophosphoryl Lipid A), 3DMPL (3-O-deacylated MPL), extracts from Aquilla, ISCOMS (see, e.g., Sjolander et al. (1998) J. Leukocyte Biol. 64:713; WO90/03184; WO96/11711; WO 00/48630; WO98/36772; WO00/41720; WO06/134423 and WO07/026190), LT/CT mutants, poly(D,L-lactide-co-glycolide) (PLG) microparticles, Quil A, interleukins, Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The immunized animal can be any animal that is capable of producing recoverable antibodies when administered an immunogen, such as, but not limited to, rabbits, mice, rats, hamsters, goats, horses, monkeys, baboons and humans. In one aspect, the host is transgenic and produces human antibodies, e.g., a mouse expressing the human immunoglobulin gene segments. U.S. Pat. Nos. 8,236,311; 7,625,559 and 5,770,429, the disclosure of each of which is incorporated herein by reference in its entirety. Lonberg et al., Nature 368 (6474): 856-859, 1994. Lonberg, N., Handbook of Experimental Pharmacology 113:49-101, 1994. Lonberg, N. and Huszar, D., Intern. Rev. Immunol., 13:65-93, 1995. Harding, F. and Lonberg, N., Ann. N.Y. Acad. Sci., 764:536-546, 1995.

The present antibodies or portions thereof can be produced by host cells transformed with DNA encoding light and heavy chains (or portions thereof) of a desired antibody. Antibodies (or portions thereof) can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding at least a portion of either or both of the light and heavy chains that is not necessary for binding, e.g., the constant region.

The present disclosure also encompasses a nucleic acid or polynucleotide encoding the present antibody, the present antigen-binding portion thereof, or a portion thereof. The nucleic acid may be expressed in a cell to produce the present antibody, the present antigen-binding portion thereof, or a portion thereof.

The nucleic acid or polynucleotide of the present disclosure may comprise at least one sequence encoding a peptide at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in any of SEQ ID NOs: 6-16, 22-27, 29-33, 40-44, 50-54, and the sequence of WTS.

The nucleic acid or polynucleotide of the present disclosure may comprise at least one sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NOs: 1-5, 17-21, 35-39, and 45-49.

The present disclosure also features expression vectors including at least one nucleic acid or polynucleotide encoding a peptide at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in any of SEQ ID NOs: 6-16, 22-27, 29-33, 40-44, 50-54, and the sequence of WTS.

The present disclosure also features expression vectors including at least one nucleic acid or polynucleotide having a sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NOs: 1-5, 17-21, 35-39, and 45-49.

Nucleic acid molecules encoding a functionally active variant of the present antibody or antigen-binding portion thereof are also encompassed by the present disclosure. These nucleic acid molecules may hybridize with a nucleic acid encoding any of the present antibody or antigen-binding portion thereof under medium stringency, high stringency, or very high stringency conditions. Guidance for performing hybridization reactions can be found in Current Protocols in Molecular Biology, John Wiley & Sons, N.Y. 6.3.1-6.3.6, 1989, which is incorporated herein by reference.

A nucleic acid or polynucleotide encoding the present antibody or antigen-binding portion thereof may be introduced into an expression vector that can be expressed in a suitable expression system, followed by isolation or purification of the expressed antibody or antigen-binding portion thereof. Optionally, a nucleic acid encoding the present antibody or antigen-binding portion thereof can be translated in a cell-free translation system. U.S. Pat. No. 4,816,567. Queen et al., Proc Natl Acad Sci USA, 86:10029-10033 (1989).

The present nucleic acids can be expressed in various suitable cells, including prokaryotic and eukaryotic cells, e.g., bacterial cells, (e.g., E. coli), yeast cells, plant cells, insect cells, and mammalian cells. A number of mammalian cell lines are known in the art and include immortalized cell lines available from the American Type Culture Collection (ATCC). Non-limiting examples of the cells include all cell lines of mammalian origin or mammalian-like characteristics, including but not limited to, parental cells, derivatives and/or engineered variants of monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells. The engineered variants include, e.g., glycan profile modified and/or site-specific integration site derivatives.

The present disclosure also provides for cells comprising the nucleic acids described herein. The cells may be a hybridoma or transfectant. The present antibody or antigen-binding portion thereof can be expressed in various cells.

When using recombinant techniques to produce, e.g., the present antibody or the antigen-binding portion thereof, the antibody or its portion can be produced intracellularly, in the periplasmic space, or directly secreted into the medium. If the antibody is produced intracellularly, the cells may be disrupted to release protein as a first step. Particulate debris, either host cells or lysed fragments, can be removed, for example, by centrifugation or ultrafiltration. Carter et al., 1992, Bio/Technology 10:163-167 describes a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems may be first concentrated using a commercially available protein concentration filter, for example, an Amicon or Millipore Pellicon ultrafiltration unit. A variety of methods can be used to isolate the antibody from the host cell.

The antibody or its portion prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

Hybridomas or other cells that produce antibodies that bind, preferably with high affinity, to SAA (e.g., SAA1) can then be subcloned and further characterized. One clone from each hybridoma or cell, which retains the reactivity of the parent cells (by ELISA), can then be chosen for making a cell bank, and for antibody purification.

Alternatively, the present antibody or antigen-binding portion thereof can be synthesized by solid phase procedures well known in the art. Solid Phase Peptide Synthesis: A Practical Approach by E. Atherton and R. C. Sheppard, published by IRL at Oxford University Press (1989). Methods in Molecular Biology, Vol. 35: Peptide Synthesis Protocols (ed. M. W. Pennington and B. M. Dunn), chapter 7. Solid Phase Peptide Synthesis, 2nd Ed., Pierce Chemical Co., Rockford, IL (1984). G. Barany and R. B. Merrifield, The Peptides: Analysis, Synthesis, Biology, editors E. Gross and J. Meienhofer, Vol. 1 and Vol. 2, Academic Press, New York, (1980), pp. 3-254. M. Bodansky, Principles of Peptide Synthesis, Springer-Verlag, Berlin (1984).

Additional antibodies (e.g., monoclonal, polyclonal, poly-specific, or mono-specific antibodies) against the SAA (e.g., SAA1) epitope recognized by 4B10 or 18A3 can be made, e.g., using a suitable method for making antibodies. In an example, a coding sequence for an epitope recognized by the 4B10 or 18A3 antibody is expressed as a C-terminal fusion with glutathione S-transferase (GST) (Smith et al., Gene 67:31-40, 1988). The fusion protein is purified on glutathione-Sepharose beads, eluted with glutathione, cleaved with thrombin (at an engineered cleavage site), and purified for immunization of rabbits. Primary immunizations are carried out with Freund's complete adjuvant and subsequent immunizations with Freund's incomplete adjuvant. Antibody titers are monitored by Western blot and immunoprecipitation analyses using the thrombin-cleaved protein fragment of the GST fusion protein. Immune sera are affinity purified using CNBr-Sepharose-coupled protein. Antiserum specificity can be determined using a panel of unrelated GST proteins.

As an alternate or adjunct immunogen to GST fusion proteins, peptides corresponding to relatively unique immunogenic regions of a polypeptide of the present disclosure can be generated and coupled to keyhole limpet hemocyanin (KLH) through an introduced C-terminal lysine. Antiserum to each of these peptides is similarly affinity purified on peptides conjugated to BSA, and specificity is tested by ELISA or Western blot analysis using peptide conjugates, or by Western blot or immunoprecipitation using the polypeptide expressed as a GST fusion protein.

Alternatively, monoclonal antibodies that specifically bind the SAA (e.g., SAA1) epitope recognized by the 4B10 or 18A3 antibody can be prepared using standard hybridoma technology (see, e.g., Kohler et al., Nature 256:495-7, 1975; Kohler et al., Eur. J. Immunol. 6:511-9, 1976; Kohler et al., Eur. J. Immunol. 6:292-5, 1976; Hammerling et al., Monoclonal Antibodies and T Cell Hybridomas, Elsevier, NY, 1981). Once produced, monoclonal antibodies can also be tested for specific recognition by Western blot or immunoprecipitation analysis. Alternatively, monoclonal antibodies can be prepared using the polypeptide of the disclosure described above and a phage display library (Vaughan et al., Nat. Biotechnol. 14:309-14, 1996).

Epitopic fragments can be generated by standard techniques, e.g., using PCR and cloning the fragment into a pGEX expression vector. Fusion proteins are expressed in E. coli and purified using a glutathione agarose affinity matrix. To minimize potential problems of low affinity or specificity of antisera, two or three such fusions are generated for each protein, and each fusion is injected into at least two rabbits. Antisera are raised by injections in a series, and can include, for example, at least three booster injections.

In order to generate polyclonal antibodies on a large scale and at a low cost, an appropriate animal species can be chosen. Polyclonal antibodies can be isolated from the milk or colostrum of, e.g., immunized cows. Polyclonal antibodies can also be isolated from the yolk of eggs from immunized chickens (Sarker et al., J. Pediatr. Gastroenterol. Nutr. 32:19-25, 2001).

### Assays

Various methods can be used to assay the antibodies or antigen-binding portions thereof to confirm their specificity for the antigen of interest and/or to study their properties. One method of conducting such assays is a sera screen assay as described in U.S. Patent Publication No. 2004/0126829. Anti-SAA (e.g., anti-SAA1) antibodies can be characterized for binding to SAA (e.g., SAA1) by a variety of known techniques. For example, in an ELISA, microtiter plates are coated with SAA (e.g., SAA1) or a fragment of SAA (e.g., SAA1) in a buffer (e.g., phosphate-buffered saline, or PBS), and then blocked with irrelevant proteins such as bovine serum albumin (BSA) diluted in PBS. Dilutions of plasma from SAA (e.g., SAA1) or a fragment of SAA (e.g., SAA1) immunized mice (or solutions containing anti-SAA (e.g., anti-SAA1) antibodies) are added to each well and incubated. The plates are washed and then incubated with a secondary antibody conjugated to an enzyme (e.g., alkaline phosphatase). After washing, the plates are developed with the enzyme's substrate (e.g., ABTS), and analyzed at a specific OD. In other embodiments, to determine if the selected monoclonal antibodies bind to unique epitopes, the antibody can be biotinylated which can then be detected with a streptavidin labeled probe. Anti-SAA (e.g., anti-SAA1) antibodies can be tested for reactivity with SAA (e.g., SAA1) by Western blotting.

Antibodies, or antigen-binding fragments, variants or derivatives thereof of the present disclosure can also be described or specified in terms of their binding affinity to an antigen. The affinity of an antibody for an antigen can be determined experimentally using any suitable method (see, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) can be made with standardized solutions of antibody and antigen, and a standardized buffer.

The present antibodies or antigen-binding portions thereof specifically bind to SAA (e.g., SAA1) with a dissociation constant (K_{D}) of less than about 10⁻⁷ M, less than about 10⁻⁸ M, less than about 9×10⁻⁹ M, less than about 8×10⁻⁹ M, less than about 7×10⁻⁹ M, less than about 6×10⁻⁹ M, less than about 5×10⁻⁹ M, less than about 4×10⁻⁹ M, less than about 3×10⁻⁹ M, less than about 2×10⁻⁹ M, less than about 1.8×10⁻⁹ M, less than about 1.5×10⁻⁹ M, less than about 1.3×10⁻⁹ M, less than about 1.2×10⁻⁹ M, less than about 10⁻⁹ M, less than about 10⁻¹⁰ M, less than about 10⁻¹¹ M, less than about 10⁻¹² M, from about 10⁻⁷ M to about 10⁻¹² M, from about 10⁻⁸ M to about 10⁻¹¹ M, from about 10⁻⁹ M to about 10⁻¹⁰ M, from about 10⁻⁸ M to about 10⁻¹² M, from about 0.9 nM to about 1.2 nM, from about 0.92 nM to about 1.15 nM, about 0.9 nM, about 0.95 nM, or about 1.1 nM. The present antibodies or antigen-binding portions thereof specifically bind to SAA (e.g., SAA1) with a half-maximal effective concentration (EC50) of less than about 10⁻⁷ M, less than about 10⁻⁸ M, less than about 9×10⁻⁹ M, less than about 8×10⁻⁹ M, less than about 7×10⁻⁹ M, less than about 6×10⁻⁹ M, less than about 5×10⁻⁹ M, less than about 4×10⁻⁹ M, less than about 3×10⁻⁹ M, less than about 2×10⁻⁹ M, less than about 10⁻⁹ M, less than about 10⁻¹⁰ M, less than about 10⁻¹¹ M, less than about 10⁻¹² M, from about 10⁻⁷ M to about 10⁻¹² M, from about 10⁻⁸ M to about 10⁻¹¹ M, from about 10⁻⁹ M to about 10⁻¹⁰ M, from about 10⁻⁸ M to about 10⁻¹² M, from about 1 nM to about 3 nM, from about 1 nM to about 2.6 nM, from about 1 nM to about 2.5 nM, from about 2 nM to about 2.6 nM, about 2.5 nM, or about 2.6 nM.

The present antibodies or fragments thereof may be administered to a subject at a dosage ranging from about 1 mg/kg body weight to about 50 mg/kg body weight, from about 2 mg/kg body weight to about 40 mg/kg body weight, from about 3 mg/kg body weight to about 30 mg/kg body weight, from about 5 mg/kg body weight to about 20 mg/kg body weight, from about 8 mg/kg body weight to about 13 mg/kg body weight, about 1 mg/kg body weight, about 2 mg/kg body weight, about 5 mg/kg body weight, about 10 mg/kg body weight, about 15 mg/kg body weight, about 20 mg/kg body weight, about 25 mg/kg body weight, about 30 mg/kg body weight, about 35 mg/kg body weight, about 40 mg/kg body weight, about 50 mg/kg body weight, about 60 mg/kg body weight, about 70 mg/kg body weight, or about 80 mg/kg body weight.

### Conditions to be Treated

Also encompassed by the present disclosure is a method of treating a cancer or a hematologic disorder in a subject, the method comprising administering to the subject an effective amount of the antibody or antigen-binding portion thereof, the polypeptide, or the composition.

The present antibodies or antigen-binding portions thereof have in vitro and in vivo therapeutic, prophylactic, and/or diagnostic utilities. For example, cells can be cultured in vitro in culture medium and contacted by the anti-SAA (e.g., anti-SAA1) antibody or fragment thereof. The antibodies or antigen-binding portions thereof can be administered to a subject, as part of an in vivo (e.g., therapeutic or prophylactic) protocol. For in vivo embodiments, the contacting step is effected in a subject and includes administering an anti-SAA (e.g., anti-SAA1) antibody or portion thereof to the subject under conditions effective to permit binding of the antibody, or portion thereof, to SAA (e.g., SAA1) in the subject. The antibodies or antigen-binding portions thereof can be administered to treat a cancer or a hematologic disorder.

The subject can be a human subject having a hematologic disorder (including a hematologic malignancy or a hematologic cancer). A hematologic disorder may refer to a abnormality involving hematopoietic cells (e.g., blood cells, including progenitor and stem cells). A hematologic malignancy or cancer may refer to a malignant abnormality involving hematopoietic cells (e.g., blood cells, including progenitor and stem cells). Hematologic cancers may refer to cancers of the blood or bone marrow.

Hematologic disorders may include myelodysplastic syndrome (MDS), age-related clonal hematopoiesis (ARCH), and hematologic malignancies or hematologic cancers.

Examples of hematologic cancers include, without limitation, lymphoma, leukemia, or multiple myeloma. Leukemias include acute myeloid leukemia, acute lymphoid leukemia, chronic myelogenous leukemia, acute lymphoblastic leukemia, chronic lymphoblastic leukemia, and chronic lymphoid leukemia.

Examples of hematological (or hematogenous) cancers include leukemias, e.g., acute leukemias (such as acute myeloid leukemia (AML), acute lymphocytic leukemia (or acute lymphoblastic leukemia, ALL), acute myelocytic leukemia, acute myelogenous leukemia and myeloblasts, promyelocytic leukemia, myelomonocytic leukemia, monocytic leukemia, and erythroleukemia), chronic leukemias (such as chronic myelomonocytic leukemia (CMML), chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Hematological disorders include, but are not limited to, myeloid disorders (including myeloid malignancies), lymphatic malignancies, malignant histiocytosis and mast cell leukemia.

In certain embodiments, myeloid disorders refer to a condition associated with a defect in the proliferation of a hematopoietic cell. In certain embodiments, myeloid disorders refer to clonal hematological diseases affecting the myeloid blood lineages, including chronic and acute conditions. Myeloid disorders include myeloproliferative neoplasms, myelodysplastic syndromes and acute myeloid leukemias. A myeloproliferative neoplasm may be primary myelofibrosis (PMF), or essential thrombocythemia (ET). Myelodysplastic syndrome may be refractory anemia with ringed sideroblasts and thrombocythemia (RARS-T). Myeloid disorders include, but are not limited to, myeloproliferative disorders (MPD), myelodysplastic syndrome (MDS), myelodysplastic/myeloproliferative disorders (MD/MPD), and acute myeloid leukemia (AML).

In some embodiments, the leukemia is acute myeloid leukemia (AML). AML is characterized as a heterogeneous, clonal, neoplastic disease that originates from transformed cells that have progressively acquired critical genetic changes that disrupt key differentiation and growth-regulatory pathways (Dohner et al., NEJM, (2015) 373:1136).

In some embodiments, the hematologic disorder is T-cell acute lymphoblastic leukemia (T-ALL), or B-cell acute lymphoblastic leukemia (B-ALL).

Lymphatic malignancies include, but are not limited to, T/NK cell tumor, B cell tumor and Hodgkin's disease.

The present antibodies or antigen-binding portions thereof and compositions may be used to treat lymphoma. Non-limiting examples of lymphoma include Hodgkin's lymphoma, non-Hodgkin's lymphoma, multiple myeloma, and immunoproliferative diseases (e.g., Epstein-Barr virus-associated lymphoproliferative diseases). Non-limiting examples of lymphoma also include, relapsed or refractory lymphoma, B-cell lymphoma, T-cell lymphoma, follicular lymphoma, double-hit lymphoma, mature B cell neoplasms, mature T cell and natural killer (NK) cell neoplasms, precursor lymphoid neoplasms, immunodeficiency-associated lymphoproliferative disorders, small lymphocytic lymphoma, Burkitt's lymphoma, etc. The lymphoma may be low-grade lymphomas, intermediate-grade lymphomas, high-grade lymphomas, low-grade lymphomas.

The cancer may be a hematological cancer or non-hematological cancer, including without limitation, lung cancer, ear, nose and throat cancer, colon cancer, melanoma, pancreatic cancer, mammary cancer, prostate cancer, breast cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods, the antibody or antigen-binding portion thereof, the polypeptide, or the composition, of the present disclosure include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the present disclosure are used to treat subjects having cancer of the breast, cervix, ovary, prostate, lung, colon and rectum, pancreas, stomach or kidney.

Sarcomas are mesenchymal neoplasms that arise in bone and soft tissues. Different types of sarcomas include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In another embodiment, the present antibody or its fragment can be used in the treatment of various disorders associated with the expression/overexpression of SAA (e.g., SAA1).

A disorder may be any condition that would benefit from treatment with the present antibody or its fragment. This includes chronic and acute disorders or diseases, including those pathological conditions that predispose the subject to the disorder in question.

### Combination Therapy

The present antibody or antigen-binding portion thereof can be administered alone or in combination with one or more other therapies or therapeutic agents (e.g., a second therapy or therapeutic agent). In some embodiments, the pharmaceutical composition comprising the anti-SAA (e.g., anti-SAA1) antibody or its fragment can further comprise a second therapeutic agent, either conjugated or unconjugated to the antibody or its fragment. In one embodiment, the second agent is another monoclonal or polyclonal antibody or antigen-binding portion thereof. In another embodiment, the second agent is a chemotherapeutic agent. In a third embodiment, the second agent is a cytotoxic or cytostatic agent. In a fourth embodiment, the second therapy may be a bone marrow transplant, or a stem cell transplant. In a fifth embodiment, the second therapy may be radiation therapy.

The second therapy or therapeutic agent may be an inhibitor of indoleamine 2,3 dioxygenase (IDO1). The IDO1 inhibitor may be indoximod (NLG8189), epacadostat (INCB024360), navoximod (GDC-0919) (NLG919), BMS-986205, PF-06840003, linrodostat (BMS-986205), MK7162, NLG802, LY-3381916, LPM-3480226, HTI-1090 (SHR9146), DN1406131, or KHK2455. See Tang et al. J. Hematol Oncol, 2021, 14:68 and Wang et al., Expert Opinion on Therapeutic Patents, 2022, Vol. 32, No. 11, 1145-1159.

The second therapy or therapeutic agent may be an agent facilitating kynurenine degradation/cleavage/breakdown or an agent facilitating a decrease in the level of kynurenine, such as kynurinase, kynureninase, kynurenine aminotransferase 1, kynurenine aminotransferase 2, and kynurenine aminotransferase 3.

The second therapy or therapeutic agent may be an inhibitor of kynurenine synthesis.

The second therapy or therapeutic agent may be an inhibitor of PD1 or an inhibitor of PD-L1. Inhibitors of PD1 may be anti-PD1 antibodies, or compounds (such as small molecule inhibitors) inhibiting PD1, including, but not limited to, pembrolizumab, nivolumab, durvalumab, cemiplimab (Libtayo), tislelizumab, sintilimab, toripalimab, and camrelizumab. Inhibitors of PD-L1 may be anti-PD-L1 antibodies, or compounds (such as small molecule inhibitors) inhibiting PD-L1, including, but not limited to, atezolizumab and avelumab.

Such combination therapy can have an additive or synergistic effect on condition parameters (e.g., severity of a symptom, the number of symptoms, or frequency of relapse).

The present anti-SAA (e.g., anti-SAA1) antibody or its fragment may be administered concurrently with the second therapy or therapeutic agent. In another specific embodiment, the second therapy or therapeutic agent is administered prior or subsequent to administration of the anti-SAA (e.g., anti-SAA1) antibody or its fragment.

### Non-Therapeutic Uses

The antibodies described herein are useful as affinity purification agents. In this process, the antibodies or fragments thereof are immobilized on a solid phase such as a Protein A resin, using methods well known in the art. The immobilized antibody or its fragment is contacted with a sample containing the SAA (e.g., SAA1) protein (or fragment thereof) to be purified, and thereafter the support is washed with a suitable solvent that will remove substantially all the material in the sample except the SAA (e.g., SAA1) protein (or fragment thereof), which is bound to the immobilized antibody. Finally, the support is washed with another suitable solvent that will release the SAA (e.g., SAA1) protein (or fragment thereof) from the antibody.

The present antibodies are also useful in diagnostic assays to detect and/or quantify SAA (e.g., SAA1) protein, for example, detecting SAA (e.g., SAA1) expression in specific cells, tissues, or serum.

The antibodies described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

### Pharmaceutical Compositions

The present disclosure provides a composition, e.g., a pharmaceutical composition, containing an antibody, or antigen-binding portion(s) thereof, of the present disclosure, formulated together with a pharmaceutically acceptable carrier. In another embodiment, the composition may contain an isolated nucleic acid encoding the present antibody or antigen-binding portion thereof, and a pharmaceutically acceptable carrier. The composition may be effective to treat a cancer or a hematologic disorder in a subject. The composition may be effective to treat MDS or leukemia in a subject. The present composition may be effective in any of the methods described herein.

Pharmaceutically acceptable carriers include any and all suitable solvents, dispersion media, coatings, antibacterial and antifungal agents, isotonic and absorption delaying agents, and the like that are physiologically compatible. Depending on the route of administration, the present antibodies (or antigen-binding portion(s) thereof) may be coated in a material to protect the antibodies (or antigen-binding portion(s) thereof) from the action of acids and other natural conditions that may inactivate the antibodies (or antigen-binding portion(s) thereof). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In certain embodiments, the present composition may include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Pharmaceutical compositions may contain the present antibody or its fragment, and optionally the second therapeutic agent as described herein.

The composition may be in the form of a solution, a suspension, an emulsion, an infusion device, or a delivery device for implantation, or it may be presented as a solid form (e.g., a dry powder) to be reconstituted with water or another suitable vehicle before use. The compositions may be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the vaccine.

The composition can be in the form of a pill, tablet, capsule, liquid, or sustained release tablet for oral administration; or a liquid for intravenous, intrathecal, subcutaneous or parenteral administration; or a polymer or other sustained release vehicle for local administration.

In one aspect, a solution of the composition is dissolved in a pharmaceutically acceptable carrier, e.g., an aqueous carrier if the composition is water-soluble. Examples of aqueous solutions include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate.

Solid formulations can be used in the present disclosure. They can be formulated as, e.g., pills, tablets, powders or capsules. For solid compositions, conventional solid carriers can be used which include, e.g., mannitol, lactose, starch, magnesium stearate, sodium saccharin, talcum, cellulose, glucose, sucrose, magnesium carbonate, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Methods well known in the art for making formulations are found, for example, in “Remington: The Science and Practice of Pharmacy” (20th ed., ed. A.R. Gennaro AR., 2000, Lippincott Williams & Wilkins, Philadelphia, PA).

In one aspect, the pharmaceutical formulations comprising compositions or nucleic acids, polypeptides, or antibodies of the present disclosure are incorporated in lipid monolayers or bilayers, e.g., liposomes. U.S. Pat. Nos. 6,110,490; 6,096,716; 5,283,185 and 5,279,833. Aspects of the present disclosure also provide formulations in which water soluble nucleic acids, peptides or polypeptides of the present disclosure have been attached to the surface of the monolayer or bilayer. For example, peptides can be attached to hydrazide-PEG-(distearoylphosphatidyl) ethanolamine-containing liposomes (see, e.g., Zalipsky, Bioconjug. Chem. 6:705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85:5-8, 1996), transmucosally, or orally. The present disclosure also provides pharmaceutical preparations in which the nucleic acid, peptides and/or polypeptides are incorporated within micelles and/or liposomes (see, e.g., Suntres, J. Pharm. Pharmacol. 46:23-28, 1994; Woodle, Pharm. Res. 9:260-265, 1992). Liposomes and liposomal formulations can be prepared according to standard methods and are also well known in the art. Akimaru, Cytokines Mol. Ther. 1:197-210, 1995. Alving, Immunol. Rev. 145:5-31, 1995. Szoka, Ann. Rev. Biophys. Bioeng. 9:467, 1980. U.S. Pat. Nos. 4,235,871; 4,501,728 and 4,837,028.

In one aspect, the compositions are prepared with carriers that will protect the peptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. U.S. Pat. No. 4,522,811.

A composition of the present disclosure can be administered by a variety of methods known in the art. As will be appreciated by the skilled artisan, the route and/or mode of administration will vary depending upon the desired results. Administration may be parenteral, intravenous, intrathecal, subcutaneous, oral, topical, local, intramuscular, intradermal, transdermal, subdermal, rectal, spinal, or epidermal. Intravenous delivery by continuous infusion is one exemplary method for administering the present antibodies.

To administer the present agent by certain routes of administration, it may be necessary to coat the agent with, or co-administer the agent with, a material to prevent its inactivation. For example, the agent may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7:27-41, 1984).

Parenteral administration can include modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Methods for preparing parenterally administrable compositions will be known or apparent to those skilled in the art and are described in detail. Bai, J. Neuroimmunol. 80:65-75, 1997. Warren, J. Neurol. Sci. 152:31-38, 1997. Tonegawa, J. Exp. Med. 186:507-515, 1997.

Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the present agent. Nanoparticulate formulations (e.g., biodegradable nanoparticles, solid lipid nanoparticles, liposomes) may be used to control the biodistribution of the present agent. Other potentially useful delivery systems include ethylene-vinyl acetate copolymer particles, osmotic pumps, intrathecal pumps, implantable infusion systems, and liposomes. The concentration of the agent in the formulation varies depending upon a number of factors, including the dosage of the drug to be administered, and the route of administration.

Sterile injectable solutions can be prepared by incorporating the present agent in the required amount in an appropriate solvent with one or a combination of ingredients enumerated above, as required, followed by sterilization microfiltration. Generally, dispersions are prepared by incorporating the present agent into a sterile vehicle that contains a basic dispersion medium and the required other ingredients from those enumerated above. In the case of sterile powders for the preparation of sterile injectable solutions, the methods of preparation include vacuum drying and freeze-drying (lyophilization) that yield a powder of the active ingredient plus any additional desired ingredient from a previously sterile-filtered solution thereof. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the antibodies may be administered once or twice weekly by subcutaneous injection or once or twice monthly by subcutaneous injection.

Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active agent calculated to produce the desired therapeutic effect in association with the required pharmaceutical carrier.

When administered orally, the present compositions may be protected from digestion. This can be accomplished either by complexing the antibody or antigen-binding portion thereof with a composition to render it resistant to acidic and enzymatic hydrolysis or by packaging the antibody or antigen-binding portion thereof in an appropriately resistant carrier such as a liposome. Means of protecting agents from digestion are well known in the art. Fix, Pharm Res. 13:1760-1764, 1996. Samanen, J. Pharm. Pharmacol. 48:119-135, 1996. U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. Sayani, Crit. Rev. Ther. Drug Carrier Syst. 13:85-184, 1996. For topical, transdermal administration, the agents are formulated into ointments, creams, salves, powders and gels. Transdermal delivery systems can also include, e.g., patches.

The present compositions can also be administered in sustained delivery or sustained release mechanisms. For example, biodegradable microspheres or capsules or other biodegradable polymer configurations capable of sustained delivery of a peptide can be included in the formulations of the invention (see, e.g., Putney, Nat. Biotechnol. 16:153-157, 1998).

For inhalation, the present compositions can be delivered using any system known in the art, including dry powder aerosols, liquids delivery systems, air jet nebulizers, propellant systems, and the like. Patton, Biotechniques 16:141-143, 1998. Also can be used in the present disclosure are product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

Compositions can be administered in a single dose treatment or in multiple dose treatments on a schedule and over a time period appropriate to the age, weight and condition of the subject, the particular composition used, and the route of administration. The frequency of administration can vary depending on any of a variety of factors, e.g., severity of the symptoms, whether the composition is used for prophylactic or curative purposes, etc. For example, in one embodiment, the composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of the present composition, e.g., the period of time over which the composition is administered, can vary, depending on any of a variety of factors, e.g., subject response, etc. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent calculated to produce the desired therapeutic effect in association with the required pharmaceutical carrier.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular agent being employed, the duration of the treatment, other drugs, agents and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian can start doses of the agents employed in the pharmaceutical composition at levels lower than that required in order to achieve the desired therapeutic effect and gradually increase the dosage until the desired effect is achieved. In general, a suitable daily dose of the present composition will be that amount of the agent which is the lowest dose effective to produce a therapeutic effect. Such an effective dose will generally depend upon the factors described above. If desired, the effective daily dose of a therapeutic composition may be administered as two, three, four, five, six or more sub-doses administered separately at appropriate intervals throughout the day, optionally, in unit dosage forms.

The data obtained from the cell culture assays and animal studies can be used in formulating a range of dosage for use in humans. In one embodiment, the dosage of such agents lies within a range of circulating concentrations that include the ED50 with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC50 (i.e., the concentration of the test agent which achieves a half-maximal inhibition of symptoms) as determined in cell culture. Sonderstrup, Springer, Sem. Immunopathol. 25:35-45, 2003. Nikula et al., Inhal. Toxicol. 4 (12): 123-53, 2000.

An exemplary, non-limiting range for a therapeutically or prophylactically effective amount of an antibody or antigen-binding portion of the present disclosure may be from about 0.001 to about 100 mg/kg body weight or more, about 0.1 to about 100 mg/kg body weight, about 0.01 to about 80 mg/kg body weight, about 0.001 to about 60 mg/kg body weight, about 0.01 to about 30 mg/kg body weight, about 0.01 to about 25 mg/kg body weight, about 0.5 to about 25 mg/kg body weight, about 0.1 to about 15 mg/kg body weight, about 0.1 to about 20 mg/kg body weight, about 10 to about 20 mg/kg body weight, about 0.75 to about 10 mg/kg body weight, about 1 to about 10 mg/kg body weight, about 2 to about 9 mg/kg body weight, about 1 to about 2 mg/kg body weight, about 3 to about 8 mg/kg body weight, about 4 to about 7 mg/kg body weight, about 5 to about 6 mg/kg body weight, about 8 to about 13 mg/kg body weight, about 8.3 to about 12.5 mg/kg body weight, about 4 to about 6 mg/kg body weight, about 4.2 to about 6.3 mg/kg body weight, about 1.6 to about 2.5 mg/kg body weight, about 2 to about 3 mg/kg body weight, or about 10 mg/kg body weight. The dosage administered to a subject may also be about 0.1 mg/kg to about 50 mg/kg, about 1 mg/kg to about 30 mg/kg, about 1 mg/kg to about 20 mg/kg, about 1 mg/kg to about 15 mg/kg, or about 1 mg/kg to about 10 mg/kg of the subject's body weight. Exemplary doses include, but are not limited to, from 1 ng/kg to 100 mg/kg. In some embodiments, a dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 16 mg/kg of the subject's body weight. WO 94/04188.

The composition is formulated to contain an effective amount of the present antibody or antigen-binding portion thereof, wherein the amount depends on the subject to be treated and the condition to be treated. In one embodiment, the present antibody or antigen-binding portion thereof is administered at a dose ranging from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 0.01 mg to about 10 g, from about 0.05 μg to about 1.5 mg, from about 10 μg to about 1 mg protein, from about 30 μg to about 500 μg, from about 40 μg to about 300 μg, from about 0.1 mg to about 200 mg, from about 0.1 mg to about 5 mg, from about 5 mg to about 10 mg, from about 10 mg to about 25 mg, from about 25 mg to about 50 mg, from about 50 mg to about 100 mg, from about 100 mg to about 500 mg, from about 500 mg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

### Articles of Manufacture

In another aspect, an article of manufacture containing materials useful for the treatment of the conditions or disorders described herein is included. The article of manufacture may be a kit. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition may be the anti-SAA (e.g., anti-SAA1) antibody or its fragment, or any other antibody or its fragment as described herein. The label on or associated with the container indicates that the composition is used for treating the condition of choice. The article of manufacture may further comprise a second container comprising a pharmaceutically acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

In one embodiment, the present disclosure provides for a kit containing an anti-SAA (e.g., anti-SAA1) antibody or antigen-binding portion thereof. Additional components of the kits may include one or more of the following: instructions for use; other reagents, a therapeutic agent, or an agent useful for coupling an antibody to a label or therapeutic agent, or other materials for preparing the antibody for administration; pharmaceutically acceptable carriers; and devices or other materials for administration to a subject.

The kit may or may not contain the second therapeutic agent as described herein. The agents can be mixed together, or packaged separately within the kit.

The kit may or may not contain at least one nucleic acid encoding anti-SAA (e.g., anti-SAA1) antibodies or fragment thereof, and instructions for expression of the nucleic acids. Other possible components of the kit include expression vectors and cells.

The present antibody or its fragment can be used in a diagnostic kit, i.e., a packaged combination of reagents in predetermined amounts with instructions for performing the diagnostic assay. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

The term “therapeutically effective amount” is an amount sufficient to treat a specified disorder or disease or alternatively to obtain a pharmacological response treating a disorder or disease.

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. The present antibodies, antigen-binding portions thereof, compositions and methods can be used in a subject such as a vertebrate, e.g., mammals and non-mammals, including human, mice, rats, guinea pigs, hamsters, dogs, cats, cows, horses, goats, sheep, pigs, monkeys, apes, gorillas, chimpanzees, rabbits, ducks, geese, chickens, amphibians, reptiles and other animals. In the context of the present disclosure, the term “subject” also encompasses tissues and cells that can be cultured in vitro or ex vivo or manipulated in vivo.

The following examples of specific aspects for carrying out the present disclosure are offered for illustrative purposes only, and are not intended to limit the scope of the present disclosure in any way.

### EXAMPLES

We recently demonstrated a crosstalk between myelodysplastic syndrome (MDS) and acute myeloid leukemia (AML) cells and osteoblasts, in which malignant cells utilize kynurenine to induce a proinflammatory state in osteoblasts, through the acute phase protein serum amyloid A1 (SAA1). In turn, SAA1 acts in a positive feedback loop on MDS and AML cells by increasing expression of IDO1, the rate-limiting enzyme for kynurenine synthesis, and potently enhancing the proliferation of MDS and AML cells, thereby enabling MDS and AML progression. This pathway is agnostic to the mutational and cytogenetic status of either disease, indicating a potent universal effect in MDS and AML. Notably, it is also active in CMML patient-derived samples that we have examined. We found that SAA1 selectively stimulates expansion of leukemic stem cells (LSCs) but not healthy hematopoietic stem cells (HSCs) by increasing proliferation and suppressing apoptosis. It also selectively prohibits differentiation of ASXL1 CRISPR-edited CD34 cells and increases their stemness.

The actions of the SAA1-IDO1 axis also relate to the effects of IDO as a key immunomodulatory enzyme. IDO1 promotes a tolerogenic milieu by expanding regulatory T cells and regulating the plasticity of the dendritic cells (DCs), among other mechanisms, and therefore favoring a tolerogenic milieu. The tumor microenvironment is a site of chronic immune activation where both immunogenic and tolerogenic processes are constitutively activated. Therapeutic success depends on killing tumor cells but also in skewing immune balance in favor of sustained immunogenic responses. Indeed, scRNAseq in patient-derived AML cells shows that SAA1 increases by 3-fold the number of LSCs and by 2-fold the number of conventional DCs (cDCs) and plasmacytoid DCs (pDCs). Notably, it also upregulates the expression of MHC class I and II antigens in LSCs and the expression of anti-phagocytosis signaling proteins such as CD47. In DCs, SAA1 upregulates the expression of IDO1.

Our studies show that SAA1 is a novel regulator of the inflammatory response and a biomarker secreted from the BM niche, that correlates with MDS development and progression to AML in patients. SAA1 levels increase with aging, mark MDS and AML patients independent of disease category, further increase with their transformation to AML and correlate with decreased survival. SAA1 potently promotes the proliferation of MDS and AML cells derived from patients independent of their cytogenetic or mutational profile without affecting proliferation of healthy CD34+ cells form healthy, age-matched subjects.

The levels of human SAA1 are higher in the bone marrow plasma of myelodisplastic syndrome (MDS) and acute myeloid leukemia (AML) patients than those of healthy subjects. Similarly, the mouse orthologue of SAA1, SAA3, is found elevated in mouse models of leukemia as well as in AML patient-derived xenograft models compared to non-leukemic wild type control or healthy CD34+transplanted mice. SAA1 levels strongly correlate with disease progression in MDS and AML patients.

SAA levels correlate with kynurenine levels. We have uncovered the relationship between both molecules: kynurenine-secreted by AML cells-acts through the serotonin receptor 1b (HTR1B) on the bone marrow (BM) niche, particularly on osteoblasts, to induce a pro-inflammatory environment that acts as a positive feedback loop to maintain leukemia progression. In particular, we have identified SAA as the osteoblast-secreted molecule able to upregulate the rate limiting enzyme for kynurenine synthesis, indoleamine 2,3-dioxygenase (IDO1) in leukemic cells. We have confirmed this mechanism in vitro using mouse and human MDS and AML cell lines, as well as ex vivo using mononuclear cells and bone marrow plasma and/or serum samples from MDS and AML patients. Moreover, we have used patient-derived xenograft models to show that SAA1 administration in vivo increases leukemia cell proliferation only in patient-derived leukemic cells, but not in healthy subjects-derived cells isolated from PDX mice. The positive feedback mechanism activated by AML-secreted

kynurenine upon HTRIB engage, triggers secretion of SAA1 by osteoblasts which in turn upregulates IDO1 expression on the AML cells, increasing their proliferation rate and perpetuating leukemia growth.

### Example 1 Effects of Polyclonal Antibody Against SAA3

We used a polyclonal anti-SAA3 (the mouse orthologue of human SAA1) antibody (pAb) to treat leukemic mice and showed that its administration decreased leukemia burden and prolonged survival in leukemic mice, despite the pAb showing less than 20% SAA-blocking ability.

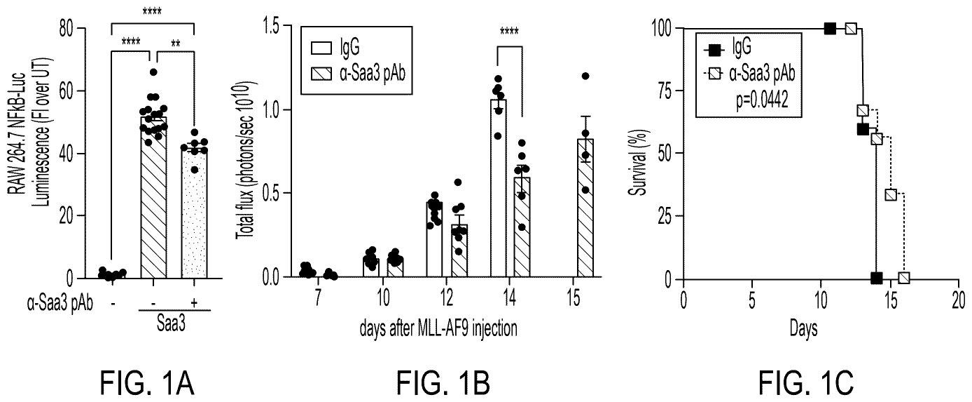

FIGS. 1A-1C show inhibition of SAA3-induced NFκB activation in RAW 264.7 cells by the anti-SAA3 polyclonal antibody. The assay aims to measure the activation of NFκB in RAW 264.7 cells (murine reporter cell line) induced by SAA3 and to evaluate the inhibitory effect of the anti-SAA3 polyclonal antibody. RAW 264.7-NFκB-Luc reporter cells contain a luciferase reporter gene under the control of NFκB response elements. Activation of NFκB leads to the production of luciferase, which can be quantified by luminescence. SAA3 protein was used to activate NFκB in the reporter cells. The anti-SAA3 polyclonal antibody (pAb) could inhibit the activation of NFκB by SAA3. Control IgG was used as a control treatment.

Specifically, Raw 264.7-NFκB-Luc cells were seeded at a density of 60,000 cells in 40 μl media/well (DMEM with 10% FBS (HiMedia)) in a 96-well plate. The plate was then incubated at 37° C. in a 5% CO₂ incubator overnight to allow cells to adhere.

For untreated control (UT), 10 μL DMEM with 0.5% FBS was added to the well. For SAA3 treatment, 5 μL SAA3 protein (150 ng/ml) was added to the well. For anti-SAA3 pAb pre-treatment, 10 μL anti-SAA3 polyclonal antibody was added to the well, before adding 5 μL SAA3 protein to the well.

The plate was incubated at 37° C. in a 5% CO₂ incubator for 2 hours to allow binding between the antibodies and antigens. Then 10 μL of the viability dye (the Cell Titer Fluor reagent) was added. The plate was rocked for 5 minutes and incubated at 37° C. in a 5% CO₂ incubator for 30 minutes. Then luminescence reading was taken with 7-amino-4-trifluoromethyl-coumarin (AFC) (excitation 405 nm/emission 495-505 m). After 3 hours, 1:1 Bright Glo Luciferase reagent (Promega) was added, and the plate was rocked for 5 minutes in dark (covered with foil) before the reading was taken.

The measured luminescence values (NFκB activity) were plotted for each treatment group.

The luminescence values of the anti-SAA3 pAb pre-treatment group were compared with the SAA3 treatment group to evaluate the inhibition effect.

FIG. 1A shows that there was a significant increase in NFκB activity in cells treated with SAA3 compared to untreated controls. Pre-treatment with anti-SAA3 pAb significantly reduced SAA3-induced NFκB activation, indicating that the antibody effectively inhibited the effect of SAA3.

FIGS. 1B-1C show the effect of anti-SAA3 polyclonal antibody on leukemia progression and survival in mice injected with MLL-AF9 cells. Bioluminescence imaging was used to quantify leukemia burden, and survival analysis was performed to evaluate the protective effect of the antibody treatment.

Specifically, 0.2×10⁶ MLL-AF9 dsRed cells (leukemia cells) were injected intravenously into C57BL/6J mice which are an animal model for leukemia. Mice were randomized into two groups: the anti-SAA3 pAb group (mice were injected with MLL-AF9 dsRed cells before being treated with the anti-SAA3 pAb) and IgG control group (mice were injected with MLL-AF9 dsRed cells before being treated with control IgG). Either the anti-SAA3 pAb or control IgG was injected intraperitoneally daily, starting 7 days after the MLL-AF9 injection. D-Luciferin substrate was injected intraperitoneally at 150 mg/kg body weight before imaging. An IVIS-spectrum optical imaging system was used to monitor leukemia progression on days 7, 14, and 15. Total flux (photons/sec) was quantified to measure leukemia burden.

Mice were monitored daily and survival time recorded. Survival curves were recorded using the Kaplan-Meier method. A log-rank test was performed to assess significance.

The total flux (bioluminescence) between the anti-SAA3 pAb group and IgG control group was compared. The reduction in leukemia burden in the anti-SAA3 pAb-treated mice was analyzed. The survival curves between the α-SAA3 pAb and IgG control groups were compared. The impact of the anti-SAA3 pAb on the survival of MLL-AF9 injected mice was also analyzed.

In FIG. 1B, the total flux (bioluminescence) was significantly higher in the IgG control group compared to the anti-SAA3 pAb group, indicating reduced leukemia burden in the antibody-treated group, which suggested that the anti-SAA3 pAb inhibits leukemia progression in the MLL-AF9 mouse model.

The survival curve in FIG. 1C indicates that mice treated with the anti-SAA3 pAb have significantly extended survivals compared to the IgG control group. The difference in survival was statistically significant (p-value: 0.0442), demonstrating the protective effect of anti-SAA3 pAb against MLL-AF9-induced leukemia.

### Example 2 Synthetic Peptide Immunization Strategy for Generating Monoclonal Antibodies

We have also generated monoclonal antibodies against human SAA1. The sequence we targeted to block SAA1 function is the Ac-RDMWRAYSDMC-amide peptide. The peptide RDMWRAYSDM in the human SAA1 protein is a conserved region between mouse SAA3 and human SAA1. We have selected the mAb clones by their ability to cross-react specifically with human SAA1 and its mouse ortholog SAA3, allowing us to test this novel mAb-based therapeutic approach in mouse models, xenotransplants and primary cells from patients. We showed that the monoclonal SAA1 antibodies suppressed proliferation of primary AML cells. This approach of targeting the niche instead of malignant cells can overcome resistance to standard of care (SOC).

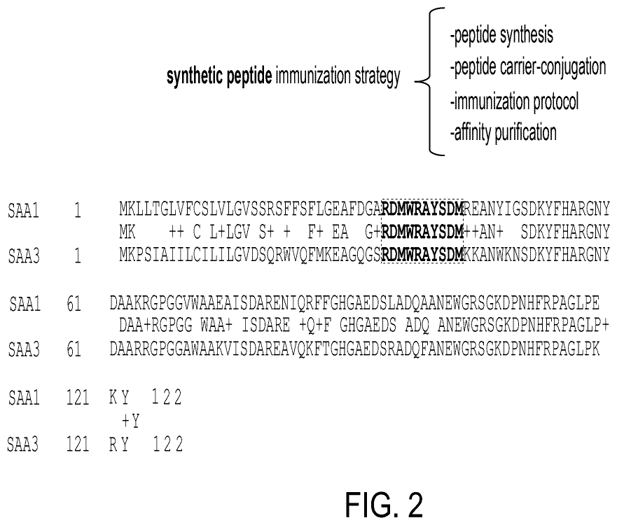

FIG. 2 shows that the synthetic peptide immunization strategy involved the following: the synthesis of a specific peptide sequence, and its conjugation to a carrier protein. Mice were immunized with the peptide to generate antibodies which were then affinity purified.

The synthesized peptide was conjugated to a carrier protein (such as KLH or BSA) to enhance the immune response. Mice were immunized with the peptide-carrier conjugate to produce antibodies. The generated antibodies were purified using affinity chromatography to isolate the specific antibodies against the synthetic peptide.

### Example 3 Evaluation of Monoclonal Antibody Subclones in Inhibiting SAA1-Induced NFκB Activation and Cell Viability

We initiated the development of blocking monoclonal antibodies against SAA1 as a means of inhibiting its potent oncogenic effects on malignant HSCs and examining its potential therapeutic activity. Utilizing a RAW 264.7 NFκB-luciferase reporter cell line, we screened 51 clones for their ability to selectively block SAA1-mediated NFκB activation. After testing, we identified seven candidate hybridomas whose unpurified supernatants demonstrated dose-dependent reduction in NFκB activity upon SAA1 challenge. Importantly, these candidate clones were specific for SAA1 and did not affect NFκB activation mediated by lipopolysaccharide (LPS)-mediated activation. We observed up to an 85% reduction of SAA1-mediated activity using supernatants from further subcloning of previously selected candidates. Moreover, cell proliferation of a human AML cell line (OCI-AML3) stimulated by SAA1 was markedly reduced in the presence of candidate antibodies, suggesting anti-proliferative activity.

We assessed the efficacy of twelve monoclonal antibody subclones in inhibiting SAA1-induced NFκβ activation in RAW 264.7 cells and their impact on cell viability in OCI-AML3 cells.

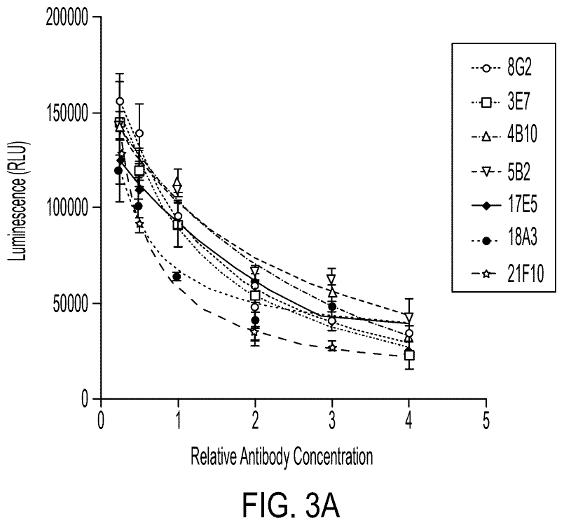

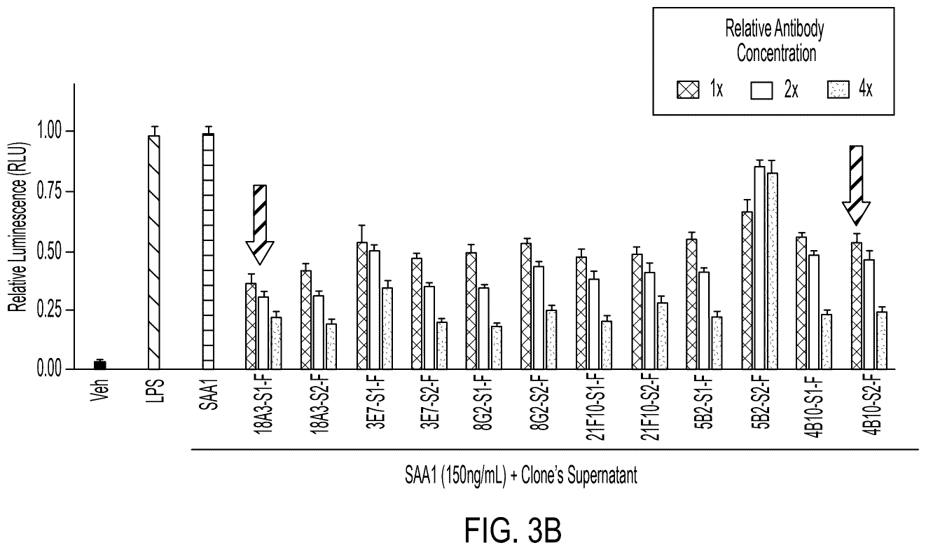

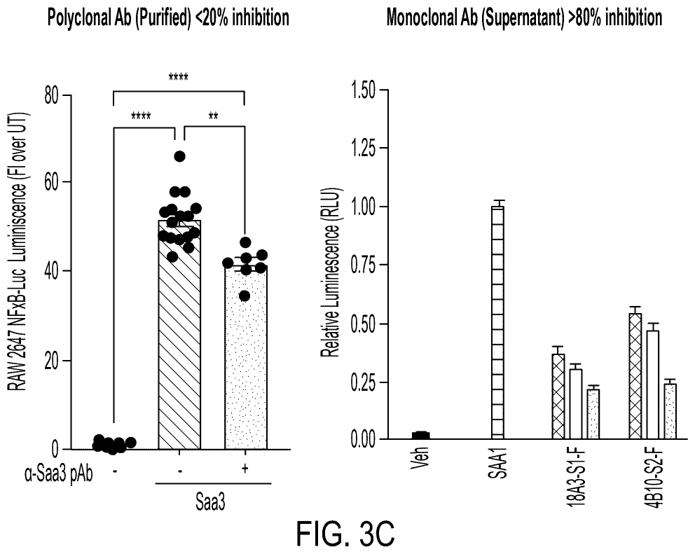

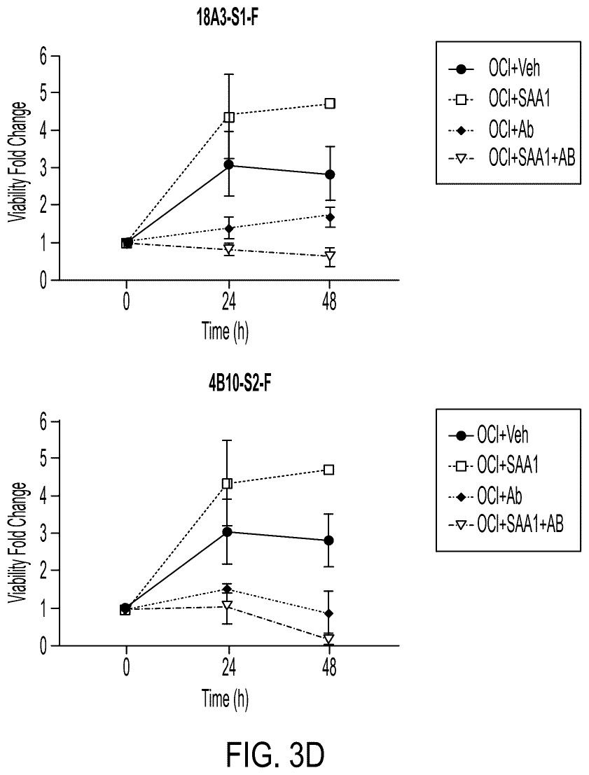

FIG. 3A shows relative luminescence units (RLU) against relative antibody concentrations for various subclones (8G2, 3E7, 4B10, 5B2, 17E5, 18A3, 21F10). Increasing antibody concentrations resulted in decreased luminescence, indicating inhibition of NFκβ activity is dose-dependent and specific.

FIG. 3B compares the relative luminescence of different monoclonal antibody supernatants at 1x, 2x, and 4× concentrations against a fixed concentration of SAA1 (150 ng/ml; Peprotech). LPS (Peprotech) was used as a positive control for NFκβ activation. Higher concentrations of antibodies result in greater inhibition of luminescence, indicating higher efficacy. Anti-SAA1 mAb subclones demonstrate dose-dependent inhibition.

FIG. 3C shows that the two selected monoclonal clones (i.e., 4B10 and 18A3) inhibited, by more than 80%, the activity of SAA1 as compared to the polyclonal antibody which only inhibited the SAA1 activity by less than 20%.

FIG. 3D shows the viability (fold change) of OCI cells treated with vehicle (“OCI+Veh”), SAA1 (“OCI+SAA1”), antibody alone (“OCI+Ab”), and antibody+SAA1 (“OCI+SAA1+AB”) over 48 hours. Monoclonal Antibodies 18A3 (subclone 18A3-S1-F) and 4B10 (subclone 4B10-S2-F) showed significant inhibition of SAA1-induced NFκβ activation and reduced cell viability in OCI cells treated with SAA1. Both subclones demonstrated high specificity and efficacy, making them suitable for targeted therapeutic applications against SAA1-induced activities.

### Example 4 Competitive Binding Assay Using Reporter Cell Line

The competitive binding assay can be used to quantify the concentration of a specific antibody in a sample (e.g., 18A3-S1-F) by taking advantage of the competition between a fixed concentration of an antigen (e.g., SAA1 antigen, fixed at 150 ng/ml) and varying concentrations of a competitive antigen (e.g., a competitive peptide varying in concentration from 80 nM to 0.625 nM) for binding to the antibody. This assay was also used to determine the dissociation constant (Kd) of the antibody. The below assay utilizes a reporter cell line to measure the resulting cellular response, which reflects the binding activity.

Competitive antigen standard solutions were prepared at the following concentrations: 80 nM, 40 nM, 20 nM, 10 nM, 5 nM, 2.5 nM, 1.25 nM, and 0.625 nM.

Raw 264.7-NFκB-Luc cells were seeded at a density of 60,000 cells in 40 μl media/well in a 96-well plate. The plate was placed at 37° C. in a 5% CO₂ incubator overnight to allow cells to adhere. 5 μL of SAA1 antigen solution (150 ng/ml) was added to each well. 10 μL of the competitive antigen standard solution was added to the respective wells. 10 μL of the purified antibody (18A3-S1-F) solution was added to each well.

The plate was incubated at 37° C. in a 5% CO₂ incubator for 2 hours to allow binding between the antibody and antigens. 10 μL of the viability dye (i.e., the Cell Titer Fluor reagent; Promega) was added, and the plate was rocked for 5 minutes and incubated at 37° C. in a 5% CO₂ incubator for 30 minutes. Then luminescence reading was taken with 7-amino-4-trifluoromethyl-coumarin (AFC) (excitation 405 nm/emission 495-505 m). After 3 hours, 1:1 Bright Glo Luciferase reagent (Promega) was added, and the plate was rocked for 5 minutes in dark (covered with foil) before the reading was taken.

The experimental groups include:
1. Empty wells
2. Medium only
3. Untreated cells
4. Cell treated only with SAA1
5. Cells treated with SAA1 and antibody
6. Cells treated with SAA1 and increasing concentrations of the competitive peptide.

The measured luminescence (NFκβ activity) was plotted against the concentrations of the competitive antigen standard solutions. A standard curve was generated, and linear regression analysis was performed to obtain the equation of the line (y=mx+b).

The equation was used to determine the concentration of the antibody in the sample by fitting the luminescence value into the equation and solving for x.

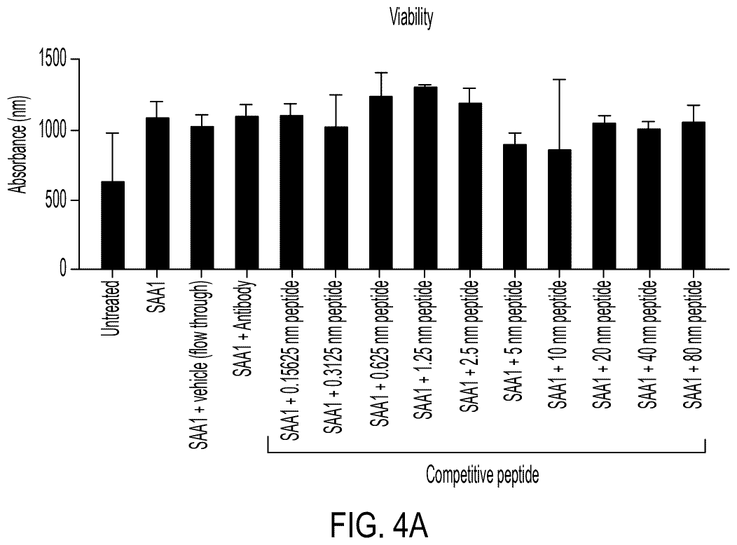

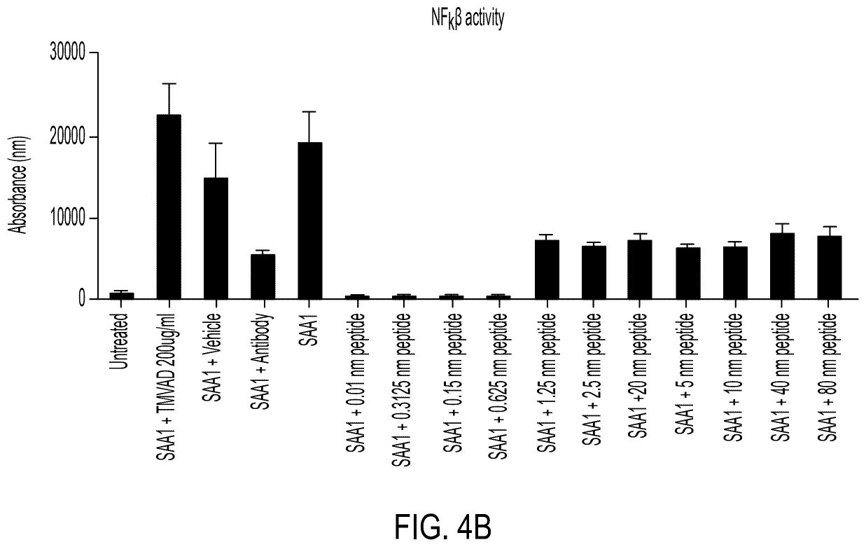

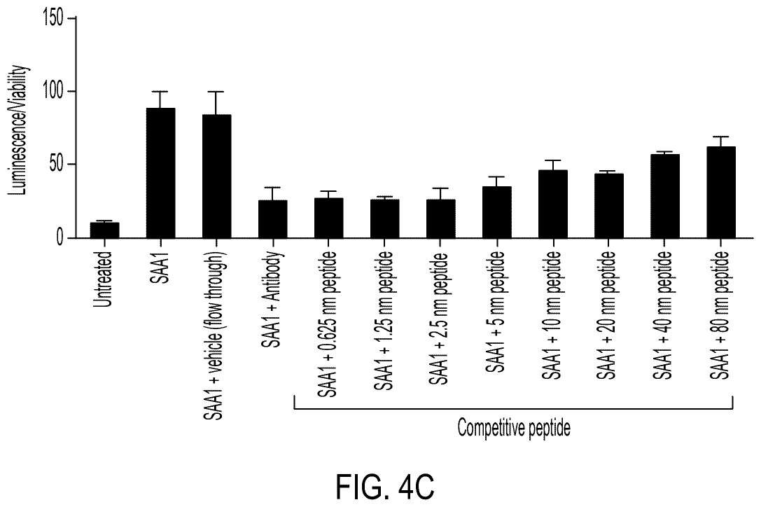

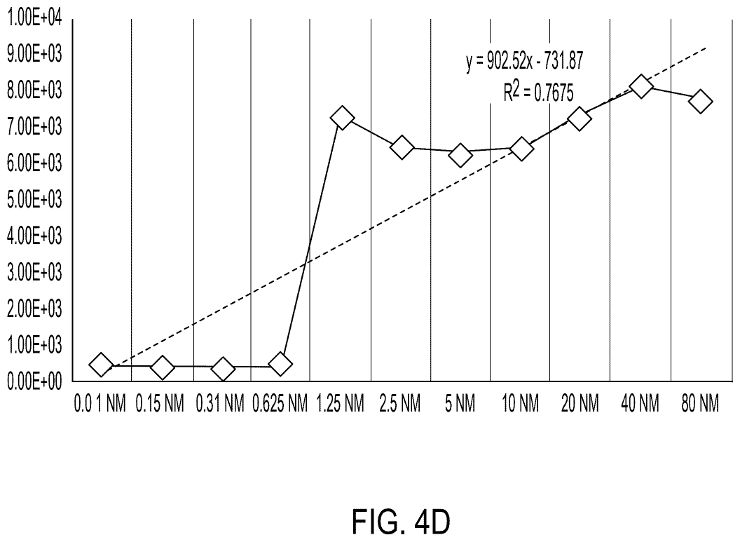

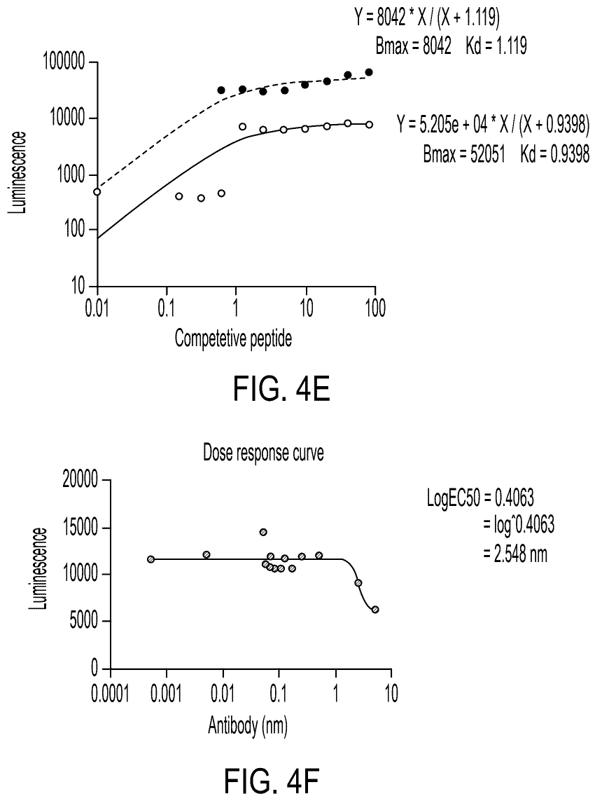

FIG. 4A shows that the viability of the cells was not affected significantly by the addition of the indicated reagents, and that the purified antibodies were not cytotoxic.

FIG. 4B shows that the antibody was blocked by the peptide at 80 nM, hence the NFκβ activity was high. At lower concentration of peptide, the antibody was able to suppress the NFκβ activity induced by SAA1. TMVAD and irrelevant peptide did not result in reduction in NFκβ activity.

FIGS. 4C and 4D show the luminescence response plotted against the concentration of the competitive peptide. The concentration of the antibody was determined using straight line equation y=mx+c. Thus, the concentration of the purified 18A3-S1-F antibody was approximately 5 nM to 6 nM.

The dissociation constant (Kd) was calculated from the competitive binding curves (FIG. 4E). The lower the Kd value, the higher the binding affinity of the antibody. The Kd values were approximately 0.9398 nM to 1.119 nM, indicating strong binding affinity.

### Example 5 Determination of EC50 for 18A3 Antibody Using NFκβ Reporter Assay

The half-maximal effective concentration (EC50) of the 18A3-S1 antibody was determined with the reporter assay. This was done by measuring the inhibition of SAA1-induced NFκβ activation at various dilutions of the antibody.

5 μL of SAA1 antigen solution (150 ng/ml) was added to each well. 10 μL of the 18A3-S1 antibody with various dilutions from 1 to 1:10000 was added to respective wells.

The LogEC50 value was determined from the dose-response curve, indicating the concentration at which the antibody achieves half-maximal inhibition of NFκβ activity. The EC50 of the 18A3-S1 antibody was calculated to be 2.548 nM (FIG. 4F), indicating the concentration at which the antibody achieves half-maximal inhibition of SAA1-induced NFκβ activation. The low EC50 value suggests that the 18A3-S1 antibody is highly effective in inhibiting NFκβ activation, making it a potent blocker of SAA1.

### Example 6 Inhibition of SAA1-Induced Proliferation by Anti-SAA1 Antibody in Cancer Cell Lines

To evaluate whether SAA1 induces proliferation in different cancer cell lines, an EdU (5-ethynyl-2′-deoxyuridine) incorporation assay was performed. This assay measures the incorporation of the nucleoside analog EdU into newly synthesized DNA, allowing the assessment of cell proliferation in response to SAA1 treatment and antibody intervention.

CUTLL1, CEM, Jurkat, Nalm6, REH, 697, BXPC3, HPAC and PANC1 are cancer cell lines. Among them, CUTLL1, CEM and Jurkat are T-cell acute lymphoblastic leukemia (T-ALL) cell lines; Nalm6, REH and 697 are B-cell acute lymphoblastic leukemia (B-ALL) cell lines.

The cancer cells were seeded at 20,000 cells per well in a 96-well plate. The medium RPMI plus 0.5% FBS was used to culture CUTLL1, CEM, Jurkat, Nalm6, REH, and 697 cell lines. The medium DMEM plus 0.5% FBS was used to culture BXPC3, HPAC, and PANC1 cell lines. The plate was incubated overnight at 37° C. in a 5% CO₂ incubator to allow cells to adhere.

The cells were treated with SAA1 protein (5 μg/ml) for 48 hours to induce proliferation. The cells were then treated with either the purified 18A3 antibody (1:1000) or PAP Fx 7 (the 18A3 antibody purified from subclone supernatant using Protein A purification kit (Invitrogen)). to assess their inhibitory effects on SAA1-induced proliferation. The controls included untreated (UT) and SAA1-only controls for comparison.

100 μl EdU (10 μM) per well was added to the wells to label newly synthesized DNA. The plate was incubated for 3 hours to allow EdU incorporation. The wells were then washed with PBS.

The cells were fixed with 4% paraformaldehyde (PFA) and permeabilized with 0.1% Triton, before being washed with PBS. 100 μl of Click-iT® EdU HCS Assay cocktail (Invitrogen) was added and incubated for 30 minutes. The cells were washed with PBS. 100 μl of HCS nuclear mask was added and incubated for 30 minutes at room temperature. The cells were washed with PBS and then resuspend in PBS.

The number of EdU-positive cells was measured using a fluorescence microscope or a high-content imaging system.

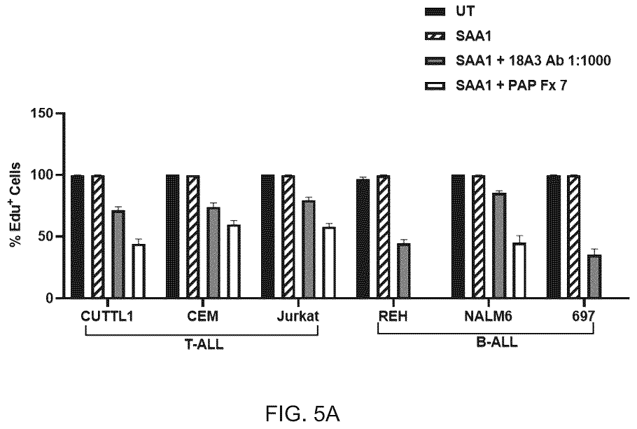

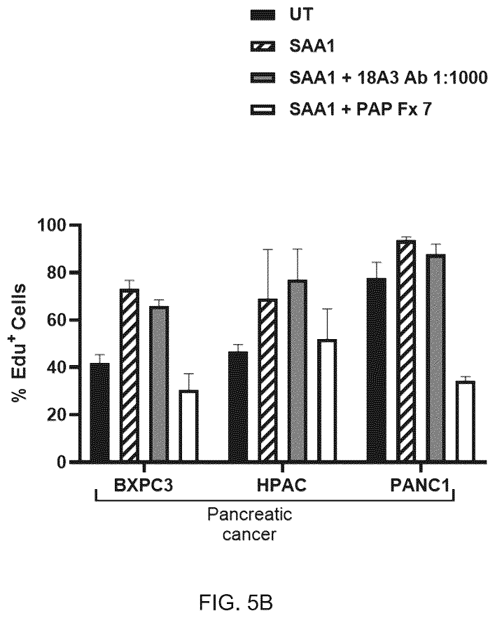

FIG. 5A shows the frequency of EdU-positive cells (indicating proliferating cells) across various T-ALL (CUTLL1, CEM, Jurkat) and B-ALL (Nalm6, REH, 697) cell lines. Treatment with the purified 18A3 antibodies reduced the frequency of EdU-positive cells across all the leukemic cell lines, indicating inhibition of SAA1-induced proliferation.

FIG. 5B shows the frequency of EdU-positive cells in pancreatic cancer cell lines (BXPC3, HPAC, PANC1) in response to SAA1 treatment. Similar to the results in the T-ALL and B-ALL cells, treatment with the purified 18A3 antibodies inhibited SAA1-induced proliferation in pancreatic cancer cell lines.

SAA1 treatment led to increased proliferation in various cancer cell lines, including T-ALL, B-ALL, and pancreatic cancer cell lines. The purified 18A3 antibodies effectively inhibited SAA1-induced proliferation across all tested cell lines.

### Example 7 Targeting SAA1 to Reduce Progression of MDS

Primary co-cultures of osteoblasts and HSCs will be set up using mesenchymal stem cells (MSCs) and patient bone marrow-mononuclear cells (BM-MNCs). Experiments will be undertaken using MSCs and BM-MNCs from MDS patients and co-cultures with osteoblasts and TET2 and DNMT3A mutated CD34+ stem cells. Samples in replicate will be treated with SAA1, then treated with either an anti-SAA1 antibody or a vehicle as a control. We will assess proliferation, apoptosis, and myeloid and erythroid differentiation rates. We will examine changes in clonality of the cells using whole exome sequencing (WES) to track changes in mutational status of the dominant clones in each population and compare the differences in clonality between co-cultures treated and untreated with anti-SAA1 antibody. Cells will be fixed and stained with erythroid and myeloid markers and will be evaluated for their off-target effects on these cell lineages as compared to untreated cells utilizing the Incell Analyzer on-site. The effects on erythroid and myeloid differentiation for each antibody will be tested at various concentrations to establish the ex vivo maximum tolerated dose (MTD) for further testing.

### Experimental Materials and Methods

### Mice

Wild type (WT) C57BL/6J (IMSR Cat # JAX: 000664, RRID: IMSR_JAX: 000664) mice were purchased from Jackson Laboratories. All other animals used in the study were bred in our mouse facility, kept in a C57BL/6J background and used between 8-10 weeks old. Male and female mice were used indistinctly.

### Cell Lines and Primary Cell Cultures

OCI-AML3 (DSMZ Cat # ACC-582, RRID: CVCL_1844) cells were acquired from the DSMZ repository.

OCI-AML3 cells as well as primary human osteoblasts were grown in MEM-Alpha 1× (Corning). All media was supplemented with 10% FBS (Gibco), except primary human osteoblasts, OCI-AML3 and HL-60 that needed 20%, 1% GlutaMAX (Gibco) and 1% antibiotic-antimycotic (Corning) and cultured at 37° C. with 5% CO_{2.}

MLL/AF9 primary cells were maintained in StemSpan medium (StemCell Technologies) containing mGM-CSF (10 ng/ml), mSCF (25 ng/ml), mIL-6 (25 ng/ml), mIL-3 (10 ng/ml), mTPO (25 ng/ml) (Prepotech) and 1% P/S.

### Leukemic Syngeneic Mouse Models and Assessment of Leukemia In Vivo Progression

All leukemia models were introduced by intravenous (i.v.) injection and transplanted into non-irradiated secondary recipient experimental animals. C57BL/6J mice were used for the MILL/AF9-dsRed leukemia model (0.2×10⁶/cells/mouse). Leukemia progression was assessed by fluorescence (MLL/AF9 dsRed) using the IVIS-Spectrum Optical Imaging System (Caliper, Perkin Elmer). Mice were shaved to reduce light attenuation.

### Immunofluorescence Staining

Tissue: after harvesting, spleen and liver were fixed o/n in 4% PFA, washed with PBS and kept on a 30% sucrose gradient for at least 16 h before OCT. For bones, fixation was done for 72 h following 7 days decalcification on 14% EDTA pH7 before sucrose gradient and OCT embedding. All tissues were cut using a Leyca cryostat, dried at RT and stored at −80° C. Sections were rehydrated in PBS for 10 min and stained with DAPI. Cells: osteoblasts were grown over 12 mm coverslips, differentiated and exposed for 30-60 min to conditioned media from OCI-AML3 cells at a 1:10 ratio, fixed in 4% PFA 15 min RT, permeabilized (PBS 0.3% Triton X-100) 15 min RT, blocked (PBS 5% donkey normal serum, 0.3% Triton X-100) and stained o/n at 4° C. with p65 (Cell Signaling Technology Cat #8242, RRID: AB_10859369) and DAPI (nuclei). Slides were mounted with anti-fade Prolong Gold (Invitrogen) mounting-medium, and images acquired on a Zeiss LSM 710 confocal microscope. Images were analyzed with ImageJ (RRID: SCR_003070) software.

While specific aspects of the invention have been described and illustrated, such aspects should be considered illustrative of the invention only and not as limiting the invention as construed in accordance with the accompanying claims. All publications and patent applications cited in this specification are herein incorporated by reference in their entirety for all purposes as if each individual publication or patent application were specifically and individually indicated to be incorporated by reference for all purposes. Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed is:

1. An anti-SAA antibody, or an antigen-binding portion thereof, comprising a heavy chain variable region (V_{H}) and a light chain variable region (V_{L}), wherein the heavy chain variable region comprises three complementarity determining regions (CDRs), CDR1, CDR2 and CDR3, having amino acid sequences about 80% to about 100% identical to the amino acid sequences set forth in SEQ ID Nos: 8, 9 and 10, respectively, or SEQ ID Nos: 42, 43 and 44, respectively, and wherein the light chain variable region comprises three CDRs, CDR1, CDR2 and CDR3, having amino acid sequences about 80% to about 100% identical to the amino acid sequences set forth in SEQ ID Nos: 24, 25 and 26, respectively, or SEQ ID Nos: 52, 53 and 54, respectively.

2. An anti-SAA antibody, or an antigen-binding portion thereof, comprising a heavy chain variable region (V_{H}), wherein the heavy chain variable region comprises three CDRs, CDR1, CDR2 and CDR3, having amino acid sequences about 80% to about 100% identical to the amino acid sequences set forth in SEQ ID Nos: 8, 9 and 10, respectively, or SEQ ID Nos: 42, 43 and 44, respectively.

3. An anti-SAA antibody, or an antigen-binding portion thereof, comprising a light chain variable region (V_{L}), wherein the light chain variable region comprises three CDRs, CDR1, CDR2 and CDR3, having amino acid sequences about 80% to about 100% identical to the amino acid sequences set forth in SEQ ID Nos: 24, 25 and 26, respectively, or SEQ ID Nos: 52, 53 and 54, respectively.

4. The antibody or antigen-binding portion thereof of claim 1, wherein the dissociation constant (K_{D}) of the antibody, or antigen-binding portion thereof, is less than about 2×10⁻⁹ M.

5. The antibody or antigen-binding portion thereof of claim 1, wherein the heavy chain variable region (V_{H}) comprises an amino acid sequence about 80% to about 100% identical to the amino acid sequences set forth in SEQ ID Nos: 6, 7, 40, or 41, and wherein the light chain variable region (V_{L}) comprises an amino acid sequence about 80% to about 100% identical to the amino acid sequences set forth in SEQ ID Nos: 22, 23, 50, or 51.

6. The antibody or antigen-binding portion thereof of claim 1, wherein the heavy chain variable region (V_{H}) comprises an amino acid sequence about 80% to about 100% identical to the amino acid sequences set forth in SEQ ID Nos: 6, 7, 40, or 41.

7. The antibody or antigen-binding portion thereof of claim 1, wherein the light chain variable region (V_{L}) comprises an amino acid sequence about 80% to about 100% identical to the amino acid sequences set forth in SEQ ID Nos: 22, 23, 50, or 51.

8. The antibody or antigen-binding portion thereof of claim 1, wherein the antibody or antigen-binding portion thereof is selected from the group consisting of: (a) a whole immunoglobulin molecule; (b) an scFv; (c) a Fab fragment; (d) an F(ab′)2; and € a disulfide linked Fv.

9. The antibody or antigen-binding portion thereof of claim 1, wherein the antibody or antigen-binding portion thereof comprises at least one constant domain selected from the group consisting of: a) an IgG constant domain; and (b) an IgA constant domain.

10. The antibody or antigen-binding portion thereof of claim 1, wherein the antibody or antigen-binding portion thereof comprises at least one human constant domain.

11. The antibody or antigen-binding portion thereof of claim 1, wherein the antibody or antigen-binding portion thereof binds to a peptide having an amino acid sequence about 80% to about 100% identical to the amino acid sequences set forth in SEQ ID Nos: 32, 33 or 34.

12. An anti-SAA antibody or an antigen-binding portion thereof, comprising a heavy chain variable region (V_{H}), wherein the heavy chain variable region comprises an amino acid sequence about 80% to about 100% identical to the amino acid sequence set forth in SEQ ID Nos: 6, 7, 40, or 41.

13. An anti-SAA antibody, or an antigen-binding portion thereof, comprising a light chain variable region (V_{L}), wherein the light chain variable region comprises an amino acid sequence about 80% to about 100% identical to the amino acid sequence set forth in SEQ ID Nos: 22, 23, 50, or 51.

14. An anti-SAA antibody, or an antigen-binding portion thereof, comprising a heavy chain variable region (V_{H}) and a light chain variable region (V_{L}), wherein the heavy chain variable region comprises an amino acid sequence about 80% to about 100% identical to the amino acid sequences set forth in SEQ ID Nos: 6, 7, 40, or 41, and wherein the light chain variable region comprises an amino acid sequence about 80% to about 100% identical to the amino acid sequences set forth in SEQ ID Nos: 22, 23, 50, or 51.

15. An anti-SAA antibody, or an antigen-binding portion thereof, comprising a heavy chain variable region (V_{H}) and a light chain variable region (V_{L}), wherein the heavy chain variable region comprises three complementarity determining regions (CDRs), CDR1, CDR2 and CDR3, encoded by nucleotide sequences about 80% to about 100% identical to the nucleotide sequences set forth in SEQ ID Nos: 3, 4 and 5, respectively, or SEQ ID Nos: 37, 38 and 39, respectively, and wherein the light chain variable region comprises three CDRs, CDR1, CDR2 and CDR3, encoded by nucleotide sequences about 80% to about 100% identical to the nucleotide sequences set forth in SEQ ID Nos: 19, 20 and 21, respectively or SEQ ID Nos: 47, 48 and 49, respectively.

16. An anti-SAA antibody, or an antigen-binding portion thereof, comprising a heavy chain variable region (V_{H}) and a light chain variable region (V_{L}), wherein the heavy chain variable region is encoded by a nucleotide sequence about 80% to about 100% identical to the nucleotide sequences set forth in SEQ ID Nos: 1, 2, 35, or 36, and wherein the light chain variable region is encoded by a nucleotide sequence about 80% to about 100% identical to the nucleotide sequences set forth in SEQ ID Nos: 17, 18, 45, or 46.

17. The antibody or antigen-binding portion thereof of claim 1, wherein the SAA is human SAA1 or mouse SAA3.

18. The antibody or antigen-binding portion thereof of claim 1, wherein the antibody or antigen-binding portion thereof is humanized or chimeric.

19. An isolated polypeptide comprising the antibody or antigen-binding portion thereof of claim 1.

20. A composition comprising the antibody or antigen-binding portion thereof of claim 1, and at least one pharmaceutically acceptable carrier.

21. A polynucleotide encoding the antibody or antigen-binding portion thereof of claim 1.

22. A vector comprising the polynucleotide of claim 21.

23. A cell comprising the vector of claim 22.

24. A method of treating a cancer or a hematologic disorder in a subject, the method comprising administering to the subject an effective amount of the antibody or antigen-binding portion thereof of claim 1.

25. The method of claim 24, wherein the hematologic disorder is acute myeloid leukemia (AML), myelodysplastic syndrome (MDS), chronic myelomonocytic leukemia (CMML), or acute lymphoid leukemia (ALL).

26. The method of claim 24, wherein the cancer is pancreatic cancer, lung cancer, liver cancer, breast cancer or colon cancer.

27. The method of claim 24, wherein the subject is a human.

28. The method of claim 24, wherein the administration is parenteral, intravenous, subcutaneous, intramuscular, transdermal, oral, topical, intrathecal, or local.

29. The method of claim 24, wherein the method further comprises administering to the subject a chemotherapeutic agent, an inhibitor of indoleamine 2,3 dioxygenase (IDO1), an inhibitor of kynurenine synthesis, an agent facilitating kynurenine breakdown, an inhibitor of PD1, an inhibitor of PD-L1, or combinations thereof.

30. An anti-SAA1 antibody or an antigen-binding portion thereof, wherein the anti-SAA1 antibody or antigen-binding portion binds to a linear amino acid sequence epitope comprising the amino acid sequence set forth in SEQ ID NO:33.

31. The anti-SAA1 antibody or antigen-binding portion of claim 30, comprising a heavy chain variable region and a light chain variable region, wherein the heavy chain variable region comprises three complementarity determining regions (CDRs), CDR1, CDR2 and CDR3, having amino acid sequences about 80% to about 100% identical to the amino acid sequences set forth in SEQ ID Nos: 8, 9 and 10, respectively, or SEQ ID Nos: 42, 43 and 44, respectively, and wherein the light chain variable region comprises three CDRs, CDR1, CDR2 and CDR3, having amino acid sequences about 80% to about 100% identical to the amino acid sequences set forth in SEQ ID Nos: 24, 25 and 26, respectively, or SEQ ID Nos: 52, 53 and 54, respectively.

32. A method of treating myelodysplastic syndrome or acute myelogenous leukemia in a subject, the method comprising administering to the subject a therapeutically effective amount of an anti-SAA1 antibody or an antigen-binding portion thereof, wherein the anti-SAA1 antibody or antigen-binding portion thereof binds to a linear amino acid sequence epitope comprising the amino acid sequence set forth in SEQ ID NO:33.

33. The method of claim 32, wherein the anti-SAA1 antibody or an antigen-binding portion thereof comprises a heavy chain variable region and a light chain variable region, wherein the heavy chain variable region comprises three complementarity determining regions (CDRs), CDR1, CDR2 and CDR3, having amino acid sequences about 80% to about 100% identical to the amino acid sequences set forth in SEQ ID NOs: 8, 9 and 10, respectively, or SEQ ID NOs: 42, 43 and 44, respectively, and wherein the light chain variable region comprises three CDRs, CDR1, CDR2 and CDR3, having amino acid sequences about 80% to about 100% identical to the amino acid sequences set forth in SEQ ID NOs: 24, 25 and 26, respectively, or SEQ ID NOs: 52, 53 and 54, respectively.


SEQUENCE LISTING (54 sequences)

SEQ ID NO: 1 - DNA, 414 bp, Mus sp.
  source 1..414: mol_type = genomic DNA
atggattggctgtggaacttgctattcctgatggcagctgcccaaagtgcccaagcacagatccagttggtgcagtctggacctgagctgaggaagcctggagagacagtcaagatctcctgcaaggcttctggatataccttcacaaactatggattgaactggattaagcaggctccaggaaagggtttacagtggatgggctggataaacacctacactggaaagccaacgtatgctgatgaattcaaggagcgatttgtcttctctttggacacctttgcccgcactgcctatttacagattaataacctcaaaaatgatgacatggcaacatatttctgtgcaaaatccctccgacgggacaggcactttgactactggggccaaggcatctctctcacagtctcctca

SEQ ID NO: 2 - DNA, 357 bp, Mus sp.
  source 1..357: mol_type = genomic DNA
cagatccagttggtgcagtctggacctgagctgaggaagcctggagagacagtcaagatctcctgcaaggcttctggatataccttcacaaactatggattgaactggattaagcaggctccaggaaagggtttacagtggatgggctggataaacacctacactggaaagccaacgtatgctgatgaattcaaggagcgatttgtcttctctttggacacctttgcccgcactgcctatttacagattaataacctcaaaaatgatgacatggcaacatatttctgtgcaaaatccctccgacgggacaggcactttgactactggggccaaggcatctctctcacagtctcctca

SEQ ID NO: 3 - DNA, 15 bp, Mus sp.
  source 1..15: mol_type = genomic DNA
aactatggattgaac

SEQ ID NO: 4 - DNA, 51 bp, Mus sp.
  source 1..51: mol_type = genomic DNA
tggataaacacctacactggaaagccaacgtatgctgatgaattcaaggag

SEQ ID NO: 5 - DNA, 30 bp, Mus sp.
  source 1..30: mol_type = genomic DNA
tccctccgacgggacaggcactttgactac

SEQ ID NO: 6 - protein, 138 aa, Mus sp.
MDWLWNLLFLMAAAQSAQAQIQLVQSGPELRKPGETVKISCKASGYTFTNYGLNWIKQAPGKGLQWMGWINTYTGKPTYADEFKERFVFSLDTFARTAYLQINNLKNDDMATYFCAKSLRRDRHFDYWGQGISLTVSS

SEQ ID NO: 7 - protein, 119 aa, Mus sp.
QIQLVQSGPELRKPGETVKISCKASGYTFTNYGLNWIKQAPGKGLQWMGWINTYTGKPTYADEFKERFVFSLDTFARTAYLQINNLKNDDMATYFCAKSLRRDRHFDYWGQGISLTVSS

SEQ ID NO: 8 - protein, 5 aa, Mus sp.
NYGLN

SEQ ID NO: 9 - protein, 17 aa, Mus sp.
WINTYTGKPTYADEFKE

SEQ ID NO: 10 - protein, 10 aa, Mus sp.
SLRRDRHFDY

SEQ ID NO: 11 - protein, 7 aa, Mus sp.
GYTFTNY

SEQ ID NO: 12 - protein, 6 aa, Mus sp.
NTYTGK

SEQ ID NO: 13 - protein, 10 aa, Mus sp.
SLRRDRHFDY

SEQ ID NO: 14 - protein, 8 aa, Mus sp.
GYTFTNYG

SEQ ID NO: 15 - protein, 8 aa, Mus sp.
INTYTGKP

SEQ ID NO: 16 - protein, 12 aa, Mus sp.
AKSLRRDRHFDY

SEQ ID NO: 17 - DNA, 381 bp, Mus sp.
  source 1..381: mol_type = genomic DNA
atggagacacattctcaggtctttgtatacatgttgctgtggttgtctggtgttgaaggagacattgtgatgacccagtctcacaaattcatgtccgcatcagtcggagacagggtcagcatcacctgcaaggccagtcaggatgtagatactgctgtagcctggtatcaacagaaaccacgacactctcctaaacttctgatttattggacatccacccgacacactggagtccctgatcgcttcacaggcagtggatctgggacagatttcactctcaccattagcaatgtgcagtctgaagacttggcagattatttctgtcagcaatttaacacctatcctctcacgttcggtactgggaccaaggtggagctgaaa

SEQ ID NO: 18 - DNA, 321 bp, Mus sp.
  source 1..321: mol_type = genomic DNA
gacattgtgatgacccagtctcacaaattcatgtccgcatcagtcggagacagggtcagcatcacctgcaaggccagtcaggatgtagatactgctgtagcctggtatcaacagaaaccacgacactctcctaaacttctgatttattggacatccacccgacacactggagtccctgatcgcttcacaggcagtggatctgggacagatttcactctcaccattagcaatgtgcagtctgaagacttggcagattatttctgtcagcaatttaacacctatcctctcacgttcggtactgggaccaaggtggagctgaaa

SEQ ID NO: 19 - DNA, 33 bp, Mus sp.
  source 1..33: mol_type = genomic DNA
aaggccagtcaggatgtagatactgctgtagcc

SEQ ID NO: 20 - DNA, 21 bp, Mus sp.
  source 1..21: mol_type = genomic DNA
tggacatccacccgacacact

SEQ ID NO: 21 - DNA, 27 bp, Mus sp.
  source 1..27: mol_type = genomic DNA
cagcaatttaacacctatcctctcacg

SEQ ID NO: 22 - protein, 127 aa, Mus sp.
METHSQVFVYMLLWLSGVEGDIVMTQSHKFMSASVGDRVSITCKASQDVDTAVAWYQQKPRHSPKLLIYWTSTRHTGVPDRFTGSGSGTDFTLTISNVQSEDLADYFCQQFNTYPLTFGTGTKVELK

SEQ ID NO: 23 - protein, 107 aa, Mus sp.
DIVMTQSHKFMSASVGDRVSITCKASQDVDTAVAWYQQKPRHSPKLLIYWTSTRHTGVPDRFTGSGSGTDFTLTISNVQSEDLADYFCQQFNTYPLTFGTGTKVELK

SEQ ID NO: 24 - protein, 11 aa, Mus sp.
KASQDVDTAVA

SEQ ID NO: 25 - protein, 7 aa, Mus sp.
WTSTRHT

SEQ ID NO: 26 - protein, 9 aa, Mus sp.
QQFNTYPLT

SEQ ID NO: 27 - protein, 6 aa, Mus sp.
QDVDTA

SEQ ID NO: 28 - skipped

SEQ ID NO: 29 - protein, 9 aa, Mus sp.
QQFNTYPLT

SEQ ID NO: 30 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 31 - protein, 122 aa, Mus sp.
MKPSIAIILCILILGVDSQRWVQFMKEAGQGSRDMWRAYSDMKKANWKNSDKYFHARGNYDAARRGPGGAWAAKVISDAREAVQKFTGHGAEDSRADQFANEWGRSGKDPNHFRPAGLPKRY

SEQ ID NO: 32 - protein, 11 aa, synthetic construct
RDMWRAYSDMC

SEQ ID NO: 33 - protein, 10 aa, Homo sapiens
RDMWRAYSDM

SEQ ID NO: 34 - protein, 11 aa, synthetic construct
  SITE 1: note = Acetylated residue
  SITE 11: note = Amidated residue
RDMWRAYSDMC

SEQ ID NO: 35 - DNA, 420 bp, Mus sp.
  source 1..420: mol_type = genomic DNA
atggactccaggctcaatttagttttccttgtccttattttaaaaggtgtccagtgtgatgtgcagctggtggagtctgggggaggcttagtgcagtctggagggtcccggaaactctcctgtgcagcctctggattcactttcagtaggtttggaatgcactgggttcgtcaggctccagagaaggggctggagtgggtcgcatacattagtcgtggcagtactaaaatctactatgcagacacagtgaagggccgattcaccatctccagagacaatcccaggaacaccctgttcctgcaaatgaccagtctaaggtctgaggacacggccatgtattactgtgcaagatctttgatctactatgattacgacggttttggttactggggccaagggactctggtcactgtctctgca

SEQ ID NO: 36 - DNA, 363 bp, Mus sp.
  source 1..363: mol_type = genomic DNA
gatgtgcagctggtggagtctgggggaggcttagtgcagtctggagggtcccggaaactctcctgtgcagcctctggattcactttcagtaggtttggaatgcactgggttcgtcaggctccagagaaggggctggagtgggtcgcatacattagtcgtggcagtactaaaatctactatgcagacacagtgaagggccgattcaccatctccagagacaatcccaggaacaccctgttcctgcaaatgaccagtctaaggtctgaggacacggccatgtattactgtgcaagatctttgatctactatgattacgacggttttggttactggggccaagggactctggtcactgtctctgca

SEQ ID NO: 37 - DNA, 15 bp, Mus sp.
  source 1..15: mol_type = genomic DNA
aggtttggaatgcac

SEQ ID NO: 38 - DNA, 51 bp, Mus sp.
  source 1..51: mol_type = genomic DNA
tacattagtcgtggcagtactaaaatctactatgcagacacagtgaagggc

SEQ ID NO: 39 - DNA, 36 bp, Mus sp.
  source 1..36: mol_type = genomic DNA
tctttgatctactatgattacgacggttttggttac

SEQ ID NO: 40 - protein, 140 aa, Mus sp.
MDSRLNLVFLVLILKGVQCDVQLVESGGGLVQSGGSRKLSCAASGFTFSRFGMHWVRQAPEKGLEWVAYISRGSTKIYYADTVKGRFTISRDNPRNTLFLQMTSLRSEDTAMYYCARSLIYYDYDGFGYWGQGTLVTVSA

SEQ ID NO: 41 - protein, 121 aa, Mus sp.
DVQLVESGGGLVQSGGSRKLSCAASGFTFSRFGMHWVRQAPEKGLEWVAYISRGSTKIYYADTVKGRFTISRDNPRNTLFLQMTSLRSEDTAMYYCARSLIYYDYDGFGYWGQGTLVTVSA

SEQ ID NO: 42 - protein, 5 aa, Mus sp.
RFGMH

SEQ ID NO: 43 - protein, 17 aa, Mus sp.
YISRGSTKIYYADTVKG

SEQ ID NO: 44 - protein, 12 aa, Mus sp.
SLIYYDYDGFGY

SEQ ID NO: 45 - DNA, 393 bp, Mus sp.
  source 1..393: mol_type = genomic DNA
atgaagttgcctgttaggctgttggtgctgatgttctggattcctgcttccagcagtgatgttttgatgacccaaactccactctccctgcctctcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 46 - DNA, 336 bp, Mus sp.
  source 1..336: mol_type = genomic DNA
gatgttttgatgacccaaactccactctccctgcctctcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 47 - DNA, 48 bp, Mus sp.
  source 1..48: mol_type = genomic DNA
agatctagtcagagcattgtacatagtaatggaaacacctatttagaa

SEQ ID NO: 48 - DNA, 21 bp, Mus sp.
  source 1..21: mol_type = genomic DNA
aaagtttccaaccgattttct

SEQ ID NO: 49 - DNA, 27 bp, Mus sp.
  source 1..27: mol_type = genomic DNA
tttcaaggttcacatgttcctccgacg

SEQ ID NO: 50 - protein, 131 aa, Mus sp.
MKLPVRLLVLMFWIPASSSDVLMTQTPLSLPLSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 51 - protein, 112 aa, Mus sp.
DVLMTQTPLSLPLSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 52 - protein, 16 aa, Mus sp.
RSSQSIVHSNGNTYLE

SEQ ID NO: 53 - protein, 7 aa, Mus sp.
KVSNRFS

SEQ ID NO: 54 - protein, 9 aa, Mus sp.
FQGSHVPPT